SEQUENCE LISTING (14 sequences)

SEQ ID NO: 1 - DNA, 1460 bp, Rhodocyclus tenuis
  misc_feature 50, 59, 881: n = unknown, m = a or c
attgaacgctggcggcatgccttacacatgcaagtcgaacggtaacgcgngggaaaccntggcgacgagtggcgaacgggtgagtaatgcatcggaacgtgccctgaagtgggggataacgtagcgaaagttacgctaataccgcatattctgtgagcaggaaagcaggggatcttaggaccttgcgctttgggagcggccgatgtcggattagctagttggtgaggtaaaagctcaccaaggcgacgatccgtagcgggtctgagaggatgatccgccacactgggactgagacacggcccagactcctacgggaggcagcagtggggaattttggacaatgggggaaaccctgatccagccatgccgcgtgagtgaagaaggccttcgggttgtaaagctctttcggcggggaagaaatggmactggctaatacctggtgtcgatgacggtacccgaagaagaagcaccggctaactacgtgccagcagccgcggtaatacgtagggtgcgagcgttaatcggaattactgggcgtaaagcgtgcgcaggcggttgtgtaagacagacgtgaaatccccgggctcaacctgggaactgcgtttgtgactgcacagctagagtttggcagaggggggtggaattccacgtgtagcagtgaaatgcgtagagatgtggaggaacaccgatggcgaaggcagccccctgggccaatactgacgctcatgcacgaaagcgtggggagcaaacaggattagataccctggtagtccacgccctaaacgatgtcaactaggtgttggtggggttaaacccattagtgccgtagctaacgcgtgaagttgaccgcctggggagtacggccgcaaggttaaaactcnaaggaattgacggggacccgcacaagcggtggatgatgtggattaattcgatgcaacgcgaaaaaccttacctacccttgacatgtcaggaatccttgagagattagggagtgcccgaaagggaacctgaacacaggtgctgcatggctgtcgtcagctcgtgtcgtgagatgttgggttaagtcccgcaacgagcgcaacccctgtcattaattgccatcattcagttgggcactctaatgagactgccggtgacaaaccggaggaaggtggggatgacgtcaagtcctcatggcccttatgggtagggcttcacacgtcatacaatggtcggtccagagggttgccaacccgcgagggggagccaatcccgcaaagccgatcgtagtccggattgcagtctgcaactcgactgcatgaagtcggaatcgctagtaatcgcggatcagcatgtcgcggtgaatacgttcccgggtcttgtacacaccgcccgtcacaccatgggagcgggttctgccagaagtagttagcctaaccgcaaggagggcgattaccacggcagggttcgtgactggggtg

SEQ ID NO: 2 - DNA, 1460 bp, Rhodocyclus tenuis
  misc_feature 50, 59: n = unknown
attgaacgctggcggcatgccttacacatgcaagtcgaacggtaacgcgngggaaaccntggcgacgagtggcgaacgggtgagtaatgcatcggaacgtgccctgaagtgggggataacgtagcgaaagttacgctaataccgcatattctgtgagcaggaaagcaggggatcttaggaccttgcgctttgggagcggccgatgtcggattagctagttggtgaggtaaaagctcaccaaggcgacgatccgtagcgggtctgagaggatgatccgccacactgggactgagacacggcccagactcctacgggaggcagcagtggggaattttggacaatgggggaaaccctgatccagccatgccgcgtgagtgaagaaggccttcgggttgtaaagctctttcggcggggaagaaatggcactggctaatacctggtgtcgatgacggtacccgaagaagaagcaccggctaactacgtgccagcagccgcggtaatacgtagggtgcgagcgttaatcggaattactgggcgtaaagcgtgcgcaggcggttgtgtaagacagacgtgaaatccccgggctcaacctgggaactgcgtttgtgactgcacagctagagtttggcagaggggggtggaattccacgtgtagcagtgaaatgcgtagagatgtggaggaacaccgatggcgaaggcagccccctgggccaatactgacgctcatgcacgaaagcgtggggagcaaacaggattagataccctggtagtccacgccctaaacgatgtcaactaggtgttggtggggttaaacccattagtgccgtagctaacgcgtgaagttgaccgcctggggagtacggccgcaaggttaaaactcaaaggaattgacggggacccgcacaagcggtggatgatgtggattaattcgatgcaacgcgaaaaaccttacctacccttgacatgtcaggaatccttgagagattagggagtgcccgaaagggaacctgaacacaggtgctgcatggctgtcgtcagctcgtgtcgtgagatgttgggttaagtcccgcaacgagcgcaacccctgtcattaattgccatcattcagttgggcactctaatgagactgccggtgacaaaccggaggaaggtggggatgacgtcaagtcctcatggcccttatgggtagggcttcacacgtcatacaatggtcggtccagagggttgccaacccgcgagggggagccaatcccgcaaagccgatcgtagtccggattgcagtctgcaactcgactgcatgaagtcggaatcgctagtaatcgcggatcagcatgtcgcggtgaatacgttcccgggtcttgtacacaccgcccgtcacaccatgggagcgggttctgccagaagtagttagcctaaccgcaaggagggcgattaccacggcagggttcgtgactggggtg

SEQ ID NO: 3 - DNA, 1478 bp, Rhodocyclus purpureus
  misc_feature 78, 541, 761, 926, 1033, 1058, 1257: n = unknown
tgaactgaagagtttgatcctggctcagattgaacgctggcggcatgccttacacatgcaagtcgaacggtaacgggnccttcgggcgccgaacgagtggcgaacgggtgagtaatgcatcggaacatgccctgaagtgggggataacgtagcgaaagttacgctaataccgcatattctgtgagcaggaaagcaggggaccttcgggccttgcgctttgggagtggccgatgtcggattagctagttggtggggtaaaagcctaccaaggcaacgatccgtagcgggtctgagaggatgatccgccacactgggactgagacacggcccagactcctacgggaggcagcagtggggaattttggacaatgggcgaaagcctgatccagccatgccgcgtgagtgaagaaggccttcgggttgtaaagctctttcggcggggaagaaatcgggtttcctaatacggaacccggatgacggtacccgaagaagaagcaccggctaactacgtgccagcagccgcggtaatacgtagggtgcnagcgttaatcggaattactgggcgtaaagcgtgcgcaggcggttgtgtaagacagacgtgaaatccccgggctcaacctgggaactgcgtttgtgactgcacagctagagtacggcagaggggggtggaattccacgtgtagcagtgaaatgcgtagagatgtggaggaacaccgatggcgaaggcagccccctgggccaatactgacgctcatgcacgnaagcgtggggagcaaacaggattagataccctggtagtccacgccctaaacgatgtcaactaggtgttggtggggttaaacccattagtgccgtagctaacgcgtgaagttgaccgcctggggagtacggcggcaaggttaaaactcaaaggaattgacgggganccgcacaagcggtggatgatgtggattaattcgatgcaacgcgaaaaaccttacctacccttgacatgtcaggaatcctgaggagactcgggagtgcccgaaagggnacctgaacacaggtgctgcatggcngtcgtcagctcgtgtcgtgagatgttgggttaagtcccgcaacgagcgcaacccttgtcattaattgccatcattcagttgggcactttaatgaaactgccggtgacaaaccggaggaaggtggggatgacgtcaagtcctcatggcccttatgggtagggcttcacacgtcatacaatggtcggtccatagggttgcnaacccgcgagggggagctaatcccagaaagccgatcgtagtccggattgcagtctgcaactcgactgcatgaagtcggaatcgctagtaatcgcggatcagcatgtcgcggtgaatacgttcccgggtcttgtacacaccgcccgtcacaccatgggagcgggttctgccagaagtagttagcctaaccgcaaggagggcgattaccacggcagcgttcgt

SEQ ID NO: 4 - DNA, 1460 bp, Rhodocyclus tenuis
  misc_feature 1045, 1315: n = unknown
attgaacgctggcggcatgccttacacatgcaagtcgaacggcagcacgggagcaatcctggtggcgagtggcgaacgggtgagtaatgcatcggaacgtgccctgaagtgggggataacgtagcgaaagttacgctaataccgcatattctgtgagcaggaaagcaggggatcgcaagaccttgcgctttgggagcggccgatgtcggattagctagttggtggggtaaaggcctaccaaggccacgatccgtagcgggtctgagaggatgatccgccacactgggactgagacacggcccagactcctacgggaggcagcagtggggaattttggacaatgggcgaaagcctgatccagccatgccgcgtgagtgaagaaggccttcgggttgtaaagctctttcggcggggaagaaattgctcaggataataccctgagtagatgacggtacccgaagaagaagcaccggctaactacgtgccagcagccgcggtaatacgtagggtgcgagcgttaatcggaattactgggcgtaaagcgtgcgcaggcggttgtgtaagacagacgtgaaatccccgggctcaacctgggaactgcgtttgtgactgcacgactagagtgtggcagaggggggtggaattccacgtgtagcagtgaaatgcgtagagatgtggaggaacaccgatggcgaaggcagccccctgggccaatactgacgctcatgcacgaaagcgtggggagcaaacaggattagataccctggtagtccacgccctaaacgatgtcaactaggtgttggtggggttaaacccattagtgccgtagctaacgcgtgaagttgaccgcctggggagtacggccgcaaggttaaaactcaaaggaattgacggggacccgcacaagcggtggatgatgtggattaattcgatgcaacgcgaaaaaccttacctacccttgacatgtcaggaatcctgaagagattcgggagtgcccgaaagggagcctgaacacaggtgctgcatggctgtcgtcagctcgtgncgtgagatgttgggttaagtcccgcaacgagcgcaacccttgtcattaattgccatcatttagttgggcactctaatgaaactgccggtgacaaaccggaggaaggtggggatgacgtcaagtcctcatggcccttatgggtagggcttcacacgtcatacaatggtcggtacagagggttgccaagccgcgaggtggagccaatcacagaaagccgatcgtagtccggattgcagtctgcaactcgactgcatgaagtcggaatcgctngtaatcgcggatcagcatgtcgcggtgaatacgttcccgggtcttgtacacaccgcccgtcacaccatgggagcgggttctgccagaagtagttagcctaaccgcaaggagggcgattaccacggcagcgttcgtgactggggtg

SEQ ID NO: 5 - DNA, 1322 bp, Rhodocyclus sp.
taccgcatattctgtgagcaggaaagcaggggatcgcaagaccttgcgctctgggagcggccgatgtcggattagctagttggtggggtaaaggcctaccaaggcgacgatccgtagcgggtctgagaggatgatccgccacactgggactgagacacggcccagactcctacgggaggcagcagtggggaattttggacaatgggcggaagcctgatccagccatgccgcgtgagtgaagaaggccttcgggttgtaaagctctttcggcggggaagaaattgcttgggttaataccctgagtagatgacggtacccgaataagaagcaccggctaactacgtgccagcagccgcggtaatacgtagggtgcgagcgttaatcggaattactgggcgtaaagcgtgcgcaggcggttttgtaagtcagatgtgaaatccccgggctcaacctgggaactgcatttgagactgcaagactggagtttggcagaggggggtggaattccacgtgtagcagtgaaatgcgtagagatgtggaggaacaccgatggcgaaggcagccccctgggccaatactgacgctcatgcacgaaagcgtggggagcaaacaggattagataccctggtagtccacgccctaaacgatgtcaactaggtgttgggagggttaaaccttttagtgccgtagctaacgcgtgaagttgaccgcctggggagtacggccgcaaggctaaaactcaaaggaattgacggggacccgcacaagcggtggatgatgtggattaattcgatgcaacgcgaaaaaccttacctacccttgacatgtcaggaatcctggagagatttgggagtgctcgcaagagagcctgaacacaggtgctgcatggctgtcgtcagctcgtgtcgtgagatgttgggttaagtcccgcaacgagcgcaacccttgtcattaattgccatcattgagttgggcactttaatgagactgccggtgacaaaccggaggaaggtggggatgacgtcaagtcctcatggcccttatgggtagggcttcacacgtcatacaatggtcggtccagagggttgccaacccgcgagggggagccaatctcagaaagccgatcgtagtccggatcgcagtctgcaactcgactgcgtgaagtcggaatcgctagtaatcgcggatcagcatgccgcggtgaatacgttcccgggtcttgtacacaccgcccgtcacaccatgggagcgggttctgccagaagtagttagcttaaccgcaaggagggcgattaccacggcagggttcgtgactggggtga

SEQ ID NO: 6 - DNA, 1460 bp, Unknown Organism
  Description of Unknown Organism Polyphosphate-accumulating organism
attaaacgctggcggcatgccttacacatgcaagtcgaacggcagcacgggggcaaccctggtggcgagtggcggacgggtgagtaatgcatcggaacgtgccctgaagtgggggataacgcagcgaaagctacgctaataccgcatattctgtgagcaggaaagcaggggatcgcaagaccttgcgctttgggagcggccgatgtcggattagctagttggtggggtaatggcctaccaaggcgacgatccgtagcgggtctgagaggatgatccgccacactgggactgagacacggcccagactcctacgggaggcagcagtggggaattttggacaatgggcggaagcctgatccagccatgccgcgtgagtgaagaaggccttcgggttgtaaagctctttcggcggggaagaaattgcttgggttaataccctgagtagatgacggtacccgaataagaagcaccggctaactacgtgccagcagccgcggtaatacgtagggtgcgagcgttaatcggaattactgggcgtaaagcgtgcgcaggcggttttgtaagtcagatgtgaaatccccgggctcaacctgggaactgcatttgagactgcaagactggagtttggcagaggggggtggaattccacgtgtagcagtgaaatgcgtagagatgtggaggaacaccgatggcgaaggcagccccctgggccaatactgacgctcatgcacgaaagcgtggggagcaaacaggattagataccctggtagtccacgccctaaacgatgtcaactaggtgttgggagggttaaaccttttagtgccgtagctaacgcgtgaagttgaccgcctggggagtacggccgcaaggctaaaactcaaaggaattgacggggacccgcacaagcggtggatgatgtggattaattcgatgcaacgcgaaaaaccttacctacccttgacatgtcaggaatcctggagagatttgggagtgctcgcaagagaacctgaacacaggtgctgcatggctgtcgtcagctcgtgtcgtgagatgttgggttaagtcccgcaacgagcgcaacccttgtcattaattgccatcattgagttgggcactttaatgagactgccggtgacaaaccggaggaaggtggggatgacgtcaagtcctcatggcccttatgggtagggcttcacacgtcatacaatggtcggtccagagggttgccaacccgcgagggggagccaatctcagaaagccgatcgtagtccggatcgcagtctgcaactcgactgcgtgaagtcggaatcgctagtaatcgcggatcagcatgccgcggtgaatacgttcccgggtcttgtacacaccgcccgtcacaccatgggagcgggttctgccagaagtagttagcctaaccgcaaggagggcgattaccacggcagggttcgtgactggggtg

SEQ ID NO: 7 - DNA, 1320 bp, Unknown Organism
  Description of Unknown Organism Polyphosphate-accumulating organism
attaaacgctggcggcatgccttacacatgcaagtcgaacggcagyacgggggcaaccctggtggcgagtggcggacgggtgagtaatgcatcggaacgtgccctgaagtgggggataacgcagcgaaagctacgctaataccgcatattctgtgagcaggaaagcaggggatcgcaagaccttgcgctttgggagcggccgatgtcggattagctagttggtggggtaatggcctaccaaggcgacgatccgtagcgggtctgagaggatgatccgccacactgggactgagacacggcccagactcctacgggaggcagcagtggggaattttggacaatgggcggaagcctgatccagccatgccgcgtgagtgaagaaggccttcgggttgtaaagctctttcggcggggaagaaattgcttgggttaataccctgagtagatgacggtacccgaataagaagcaccggctaactacgtgccagcagccgcggtaatacgtagggtgcgagcgttaatcggaattactgggcgtaaagcgtgcgcaggcggttttgtaagtcagatgtgaaatccccgggctcaacctgggaactgcatttgagactgcaagactggagtttggcagaggggggtggaattccacgtgtagcagtgaaatgcgtagagatgtggaggaacaccgatggcgaaggcagccccctgggccaatactgacgctcatgcacgaaagcgtggggagcaaacaggattagataccctggtagtccacgccctaaacgatgtcaactaggtgttgggagggttaaaccttttagtgccgtagctaacgcgtgaagttgaccgcctggggagtacggccgcaaggctaaaactcaaaggaattgacggggacccgcacaagcggtggatgatgtggattaattcgatgcaacgcgaaaaaccttacctacccttgacatgtcaggaatcctggagagatttgggagtgctcgcaagagaacctgaacacaggtgctgcatggctgtcgtcagctcgtgtcgtgagatgttgggttaagtcccgcaacgagcgcaacccttgtcattaattgccatcattgagttgggcactttaatgagactgccggtgacaaaccggaggaaggtggggatgacgtcaagtcctcatggcccttatgggtagggcttcacacgtcatacaatggtcggtccagagggttgccaacccgcgagggggagccaatctcagaaagccgatcgtagtccggatcgcagtctgcaactcgactgcgtgaagtcggaatcgctagtaat

SEQ ID NO: 8 - DNA, 1459 bp, Unknown Organism
  Description of Unknown Organism Polyphosphate-accumulating organism
attaaacgctgcggcatgccttacacatgcaagtcgaacggcagcacgggggcaaccctggtggcgagtggcggacgggtgagtaaagcatcggaacgtatcctggagtgggggataacgtagcgaaagttacgctaataccgcatattctgtgagcaggaaagcaggggatcgcaagaccttgcgctctgggagcggccgatgtcggattagctagttggtggggtaaaggcctaccaaggcgacgatccgtagcgggtctgagaggatgatccgccacactgggactgagacacggcccagactcctacgggaggcagcagtggggaattttggacaatgggcgcaagcctgatccagccatgccgcgtgagtgaagaaggccttcgggttgtaaagctctttcgrcggggaagaaattgcacgggttaataccctgtgtagatgacggtacccgaataagaagcaccggctaactacgtgccagcagccgcggtaatacgtagggtgcgagcgttaatcggaattactgggcgtaaagcgtgcgcaggcggtttggtaagtcagatgtgaaatccccgggctcaacctgggaactgcatttgagactgccaggctggagtttggcagaggggggtggaattccacgtgtagcagtgaaatgcgtagagatgtggaggaacaccgatggcgaaggcagccccctgggccaatactgacgctcatgcacgaaagcgtggggagcaaacaggattagataccctggtagtccacgccctaaacgatgtcaactaggtgttgggagggttaaaccttttagtgccgtagctaacgcgtgaagttgaccgcctggggagtacggccgcaaggctaaaactcaaaggaattgacggggacccgcacaagcggtggatgatgtggattaattcgatgcaacgcgaaaaaccttacctacccttgacatgtcaggaatcctgaagagatttgggagtgctcgcaagagagcctgaacacaggtgctgcatggctgtcgtcagctcgtgtcgtgagatgttgggttaagtcccgcaacgagcgcaacccttgtcattaattgccatcatttagttgggcactttaatgagactgccagtgacaaaccggaggaaggtggggatgacgtcaagtcctcatggcccttatgggtagggcttcacacgtcatacaatggtcggtccagagggttgccaacccgcgagggggagccaatctcagaaagccgatcgtagtccggatcgcagtctgcaactcgactgcgtgaagtcggaatcgctagtaatcgcggatcagcatgtcgcggtgaatacgttcccgggtcttgtacacaccgcccgtcacaccatgggagcgggttctgccagaagtagttagcctaaccgcaaggagggcgattaccacggcagggttcgtgactggggtg

SEQ ID NO: 9 - DNA, 1426 bp, Unknown Organism
  Description of Unknown Organism Polyphosphate-accumulating organism
attaaacgctgcggcatgccttacacatgcaagtcgaacggcagcacgggggcaaccctggtggcgagtggcgaacgggtgagtaaagcatcggaacgtgccctggaatgggggataacgtagcgaaagttacgctaataccgcatattctgtgagcaggaaagcaggggatcgcaagaccttgcgttcgaggaacggccgatgtcggattagctagttggtggggtaaaagcctaccaaggcaacgatccgtagcgggtctgagaggatgatccgccacactggaactgagacacggtccagactcctacgggaggcagcagtggggaattttggacaatgggcgcaagcctgatccagccatgccgcgtgagtgaagaaggccttcgggttgtaaagctctttcggccgggaagaaatcgcacgggtaaataccctgtgtggatgacggtaccggaataagaagcaccggctaactacgtgccagcagccgcggtaatacgtagggtgcgagcgttaatcggaattactgggcgtaaagcgtgcgcaggcggtttggtaagtcagatgtgaaatccccgggctcaacctgggaactgcatttgagactgccaagctggagtttggcagaggggggtggaattccacgtgtagcagtgaaatgcgtagagatgtggaggaacaccgatggcgaaggcagccccctgggccaatactgacgctcatgcacgaaagcgtggggagcaaacaggattagataccctggtagtccacgccctaaacgatgtcaactaggtgttgggagggttaaaccttttagtgccgtagctaacgcgtgaagttgaccgcctggggagtacggccgcaaggctaaaactcaaaggaattgacggggacccgcacaagcggtggatgatgtggattaattcgatgcaacgcgaaaaaccttacctacccttgacatgtcaggaatcccggagagatttgggagtgctcgcaagagaacctgaacacaggtgctgcatggctgtcgtcagctcgtgtcgtgagatgttgggttaagtcccgcaacgagcgcaacccttgtcattaattgccatcattcagttgggcactttaatgagactgccggtgacaaaccggaggaaggtggggatgacgtcaagtcctcatggcccttatgggtagggcttcacacgtcatacaatggtcggtccagagggttgccaacccgcgagggggagccaatcccagaaagccgatcgtagtccggatcgcagtctgcaactcgactgcgtgaagtcggaatcgctagtaatcgcggatcagcatgccgcggtgaatacgttcccgggtcttgtacacaccgcccgtcacaccatgggagcgggttctgccagaagtagttagcctaaccgcaaggagg

SEQ ID NO: 10 - DNA, 1485 bp, Propionibacter pelophilus
ggctcagattgaacgctggcggcatgccttacacatgcaagtcgaacggcagcatgggtgcttgcacctgatggcgagtggcgaacgggtgagtaatgcatcggaacgtacccggaagtgggggataacgtagcgaaagttacgctaataccgcatattctgtgagcaggaaagagggggatcgcaagacctctcgctttcggagcggccgatgtcggattagctagttggtggggtaaaggcctaccaaggcgacgatccgtagcgggtctgagaggatgatccgccacactgggactgagacacggcccagactcctacgggaggcagcagtggggaattttggacaatgggcgcaagcctgatccagccatgccgcgtgagtgaagaaggccttcgggttgtaaagctctttcggtcgggaagaaatggcacgctctaacatagcgtgttgatgacggtaccgacataagaagcaccggctaactacgtgccagcagccgcggtaatacgtagggtgcgagcgttaatcggaattactgggcgtaaagcgtgcgcaggcggttgtgtaagtcagaggtgaaatccccgggctcaacctgggaatggcctttgagactgcacggctagagtgtgacagaggggggtagaattccacgtgtagcagtgaaatgcgtagagatgtggaggaataccgatggcgaaggcagccccctgggttactactgacgctcatgcacgaaagcgtggggagcaaacaggattagataccctggtagtccacgccctaaacgatgtcaactggatgttgggagggttaaacctcttagtgtcgtagctaacgcgtgaagttgaccgcctggggagtacggccgcaaggctaaaactcaaaggaattgacggggacccgcacaagcggtggatgatgtggattaattcgatgcaacgcgaaaaaccttacctacccttgacatgtcaggaatccttgagagattgaggagtgcccgaaagggagcctgaacacaggtgctgcatggctgtcgtcagctcgtgtcgtgagatgttgggttaagtcccgcaacgagcgcaacccttgtcgttaattgccatcattaagttgggcactttaatgagactgccggtgacaaaccggaggaaggtggggatgacgtcaagtcctcatggcccttatgggtagggcttcacacgtcatacaatggtcggttcagagggttgccaacccgcgagggggagccaatctcagaaagccgatcgtagtccggattgcagtctgcaactcgactgcatgaagtcggaatcgctagtaatcgcggatcagcatgccgcggtgaatacgttcccgggtcttgtacacaccgcccgtcacaccatgggagcgggttctgccagaagtaggtagcctaaccgcaaggagggcgcttaccacggcggggttcgtgactggggtgaagtcgtaacaaggta

SEQ ID NO: 11 - DNA, 24 bp, Artificial Sequence
  Description of Artificial Sequence Oligonucleotide probe/primer
ccgtcatctacwcagggtattaac

SEQ ID NO: 12 - DNA, 18 bp, Artificial Sequence
  Description of Artificial Sequence Oligonucleotide probe/primer
ccctctgccaaactccag

SEQ ID NO: 13 - DNA, 21 bp, Artificial Sequence
  Description of Artificial Sequence Oligonucleotide probe/primer
gttagctacggcactaaaagg

SEQ ID NO: 14 - DNA, 22 bp, Artificial Sequence
  Description of Artificial Sequence Oligonucleotide probe/primer
aggattcctgacatgtcaaggg

(19) United States
(12) Patent Application Publication
(10) Pub. No.: US 2003/0170654 A1
(43) Pub. Date: Sep. 11, 2003

(54) Probes and primers for the detection of polyphosphate accumulating organisms in wastewater

(76) Inventors: Gregory Robert Crocetti, Queensland (AU); Gene William Tyson, Queensland (AU); Philip Hugerholtz, Queensland (AU); Linda Louise Blackall, Queensland (AU)

Correspondence Address: BURNS DOANE SWECKER & MATHIS L L P, POST OFFICE BOX 1404, ALEXANDRIA, VA, 22313-1404, US

(21) Appl. No.: 10/168,337

(22) PCT Filed: Dec. 28, 2000
(86) PCT No.: PCT/AU00/01611

(51) Int. Cl. C12Q 1/68; C07H 21/04

(52) U.S. Cl. 435/6; 536/24.3

(57) ABSTRACT

The present invention relates to the identification of polyphosphate accumulating organisms that are capable of biologically removing phosphorus from wastewater. Specifically, the invention provides oligonucleotide probes or primers for detecting a polyphosphate accumulating organism in a sample. These oligonucleotides have a sequence of at least 12 nucleotides that is unique to 16S rDNA of polyphosphate accumulating organisms. The invention further provides methods for detecting polyphospbate accumulating organisms.

### TECHNICAL FIELD

This invention relates to the identification of polyphosphate accumulating organisms that are capable of biologically removing phosphorus from wastewater. In particular, the invention relates to a method for rapidly assessing the presence or numbers of such organisms in a wastewater sample, their numbers being indicative of the phosphorus-removing capacity of the wastewater microbial community.

### INTRODUCTION

Domestic wastewater is typically treated by an activated sludge process designed to remove nutrients such as carbon (C), nitrogen (N) and phosphorus (P) from the wastewater in order to prevent global eutrophication (Metcalf and Eddy, 1991). This process is biologically mediated, relying on microorganisms to take up such nutrients from the wastewater for incorporation into growing and dividing cells, or to volatilise the nutrients. For instance, nitrate can be reduced to dinitrogen gas and dissipated into the atmosphere (Seviour and Blackall, 1999).

Microorganisms in the activated sludge process grow as flocs—three-dimensional agglomerates about 100 μm in diameter. These flocs can be separated from the treated wastewater by gravity sedimentation, but such separation processes, however, are prone to failures costing hundreds of thousands of dollars in remedial action each year in Australia alone. There are two main reasons for these failures:

1. Solids separation problems where the biomass does not separate from the treated water. Bulking occurs when filamentous bacteria form bridges between flocs precluding their settlement and compaction. Overgrowth of the biomass by hydrophobic filamentousbacteria which are selectively floated on the liquid surfaces also leads to lack of clear separation of the biomass into one fraction and the liquid into another. This latter problem is called foaming.

2. Loss of appropriate active microbial community. Particular populations of microorganisms within the wastewater microbial community are responsible for P or N uptake and removal. If these populations drop below a certain number then P and N removal will drop accordingly.

The removal of P from wastewater can be achieved by chemical precipitation or by biological mechanisms in a process called enhanced biological phosphorus removal (EBPR). The basic configuration of an EBPR activated sludge plant has the influent wastewater going into an anaerobic zone where it is mixed with the returned microbial biomass from the secondary clarifier to form the so-called mixed liquor. This mixed liquor then flows into an aerobic zone after which the biomass is separated from the treated wastewater in the secondary clarifier. Polyphosphate accumulating organisms (PAOs) (van Loosdrecht et al., 1997) are selectively enriched in these systems and excessive phosphate accumulation occurs in the aerobic zone. Removal of a portion of the growing biomass (waste activated sludge) results in the net removal of P from the wastewater.

Empirical experience over the last 30-40 years of EBPR operation has permitted plant operators to more successfully conduct EBPR processes (Hartley & Sickerdick, 1994). However, despite this experience, the study of EBPR microbiology remains important as EBPR processes do fail intermittently. By the time a wastewater treatment plant operator has detected an EBPR process failure, which is done by monitoring P levels, the change in the microbial community leading to this failure will already have been underway for a period of time and in fact the PAOs may have reached such low levels that they have no ability to compete in the microbial community. Moreover, the PAOs have not been unambiguously identified and the biochemical pathways for P removal are unknown. Researchers have constructed biochemical models that accommodate the gross chemical transformations observed in EBPR processes (Comeau et al., 1986; Wentzel et al., 1991).

There have been many investigations attempting to match the metabolic performance of bacterial isolates with the biochemical model suggested for EBPR. These have concentrated mostly on isolates of the genus Acinetobacter because members of this genus are easily isolated from EBPR sludges (Fuhs & Chen, 1975; Kerdachi & Healey, 1987; Wentzel et al., 1988) and some isolates show some characteristics that may be important to EBPR (Deinema et al., 1985; Streichan et al., 1990). However, evidence indicating that Acinetobacter may not be responsible for EBPR includes pure culture performances not correlating with biological models (Bond, 1997; Tandoi et al., 1998), and analyses of EBPR bacterial communities indicating that Acinetobacter are not present in high enough numbers to account for EBPR (Bond, 1997; Cloete & Steyn, 1987; Kampfer et al., 1996; Melasniemi et al., 1999; Wagner et al., 1994). Investigations of other EBPR-associated microorganisms are limited, although there has been some interest in Gram positive bacteria such as Microlunatus phosphovorus (Nakamura et al., 1995; Ubukata, 1994), the Gram negative Lampropedia (Stante et al., 1997) and the Actinobacteria and α-Proteobacteria (Kawaharasaki et al., 1999). However, there is no general consensus that these bacteria are examples of PAOs and indeed Mino et al. (1998) concluded that rather than being a single dominant PAO several different bacterial groups could be important. The isolation of putative PAOs is hampered by the lack of an easy method to use the P removal phenotype in isolation strategies.

Knowledge of the microorganisms responsible for enhancing biological phosphorus removal from wastewater is desirable for efficient management of ,treatment systems. It is also desirable to be able to rapidly determine the numbers of such organisms in order to assess the phosphorus-removing capacity of a microbial community, much like an early warning system should the EBPR process begin to fail.

### SUMMARY OF THE INVENTION

An object of the invention is to provide oligonucleotides that can be used to detect polyphosphate accumulating organisms in a sample. Further objections of the invention are to provide methods of detecting, or quantifying the level of the foregoing organisms in a sample.

According to a first embodiment of the invention, there is provided an oligonucleotide probe for detecting a polyphosphate accumulating organism in a sample, said oligonucleotide having a sequence of at least 12 nucleotides that is unique to 16S rDNA of polyphosphate accumulating organisms.

According to a second embodiment of the invention, there is provided an oligonucleotide primer for PCR amplification of DNA of a polyphosphate accumulating organism, said primer having a sequence of at least 12 nucleotides that is unique to 16S rDNA of polyphosphate accumulating organisms.

According to a third embodiment of the invention, there is provided a primer pair for PCR amplification of 16S rDNA of a polyphosphate accumulating organism, said primer pair comprising:

(a) a first oligonucleotide of at least 12 nucleotides having a sequence selected from one strand of said 16S rDNA; and

(b) a second oligonucleotide of at least 12 nucleotides having a sequence selected from the other strand of said 16S rDNA downstream of said first oligonucleotide sequence; wherein at least one of said first and second oligonucleotides has a sequence that is unique to 16S rDNA of polyphosphate accumulating organisms.

According to a fourth embodiment of the invention, there is provided a method of detecting cells of a polyphosphate accumulating organism in a sample, said method comprising the steps of:

(a) treating cells in said sample to fix cellular contents;

(b) contacting said fixed cells from step (a) with a labeled oligonucleotide probe under conditions which allow said probe to hybridize with 16S rRNA within said fixed cell, wherein said probe is an oligonucleotide according to the first embodiment;

(c) removing unhybridized probe from said fixed cells; and

(d) detecting said labeled probe-RNA hybrid.

According to a fifth embodiment of the invention, there is provided a method of detecting a polyphosphate accumulating organism in a sample, said method comprising the steps of:

(a) obtaining nucleic acid from cells of said organism;

(b) contacting nucleic acid from step (a) with a labeled or immobilised oligonucleotide probe under conditions which allow said probe to hybridize to 16S nucleic acid molecules, wherein said probe is an oligonucleotide according to the first embodiment;

(c) if necessary, separating unhybridized probe and labeled probe-nucleic acid hybrid; and

(d) detecting said labeled probe-nucleic acid hybrid.

According to a sixth embodiment of the invention, there is provided a method of detecting a polyphosphate accumulating organism in a sample, said method comprising the steps of:

(a) lysing cells of the organism to release genomic DNA;

(b) contacting denatured genomic DNA from step (a) with a primer pair according to the third embodiment;

(c) amplifying 16S rDNA of said organism by cyclically reacting said primer pair with said rDNA to produce an amplification product; and

(d) detecting said amplification product.

In other embodiments of the invention, there are provided methods of quantitating the number of polyphosphate accumulating organisms in a sample. The invention further provides a method of identifying oligonucleotide probes suitable for the detection or quantitation of polyphosphate accumulating organisms.

### BRIEF DESCRIPTION OF THE DRAWINGS

FIG. 1 is a phylogenetic tree constructed from near complete 16S rDNA sequences derived from a variety of sludges and sequences from publically accessible databases.

FIG. 2 shows fluorescence in situ hybridization micrographs of mixed liquors from sequencing batch reactors.

FIG. 3 is an alignment of 16S rDNA sequences.

FIG. 4 shows the relationship between sludge P content (% of dry mass) and % cells binding all three PAO probes of Table 4 as a percentage of the EUB338-probe positive cells.

### BEST MODE AND OTHER MODES OF CARRYING OUT THE INVENTION

The following abbreviations are used hereafter:

| | |
|---|---|
| bp | base pair |
| C | carbon |
| CH | carbohydrates |
| EBPR | enhanced biological phosphorus removal |
| FID GC | flame ionization detector gas chromatography |
| FISH | fluorescence in situ hybridization |
| HRT | hydraulic retention time |
| N | nitrogen |
| P | phosphorus |
| PAO | polyphosphate accumulating organism |
| PCR | polymerase chain reaction |
| PHA | polyhydroxyalkanoate |
| Pns | non-soluble phosphate |
| Psol | soluble orthophosphate |
| Pt | total orthophosphate |
| rDNA | ribosomal DNA |
| RFLP | restriction fragment length polymorphism |
| rRNA | ribosomal RNA |
| SBR | sequencing batch reactor |
| SEP | San Francisco Southeast Water Pollution Control Plant |
| SRT | solids retention time |
| Tm | melting temperature |
| Td | dissociation temperature |
| TSS | total suspended solids |
| VSS | volatile suspended solids |

The one-letter code for nucleotides in DNA conforms to the IUPAC-IUB standard described in Biochemical Journal 219, 345-373 (1984).

The term “comprise” and variants of the term such as “comprises” or “comprising” are used herein to denote the inclusion of a stated integer or stated integers but not to exclude any other integer or any other integers, unless in the context or usage an exclusive interpretation of the term is required.

The entire content of each publication or article cited hereafter is incorporated into the description by cross-reference. However, the cross-referencing of an article or publication does not mean that the article or publication constitutes common general knowledge.

The present inventors have utilized a laboratory scale sequencing batch reactor (SBR) to generate sludges enriched in polyphosphate accumulating organisms (PAOs) and have prepared 16S rDNA clone libraries from these and other sludges. Evidence is provided that the characterized 16S rDNA sequences derive from PAOs belonging to the bacterial subdivison β-2 Proteobacteria, and that the 16S rRNA sequences are most closely related to Rhodocyclus spp. and Propionibacter pelophilus. The inventors have surprisingly found that there are unique sequences in the 16S rDNA of PAOs, which sequences can be used as sequences for oligonucleotide probes or primers. The probes an be used in various hybridisation techniques for detecting PAOs and the primers for PCR amplification of DNA of such organisms.

A 16S rRNA- or rDNA-directed oligonucleotide probe or primer of the first embodiment of the invention typically has a length of about 12 to 50 nucleotides. A preferred length is 15 to 25 nucleotides. Particularly preferred oligonucleotides of the first embodiment are as follows:

5′-CCGTCATCTACWCAGGGTATTAAC-3′
5′-CCCTCTGCCAAACTCCAG-3′
5′-GTTAGCTACGGCAGTAAAAGG-3′

The invention also provides probes or primers for detecting organisms closely related to PAOs. With such primers mismatches at the 5′ end of the primer are permissible. A preferred primer for detecting organisms closely related to PAOs has the following sequence:

5′-AGGATTCCTGACATGTCAAGGG-3′.

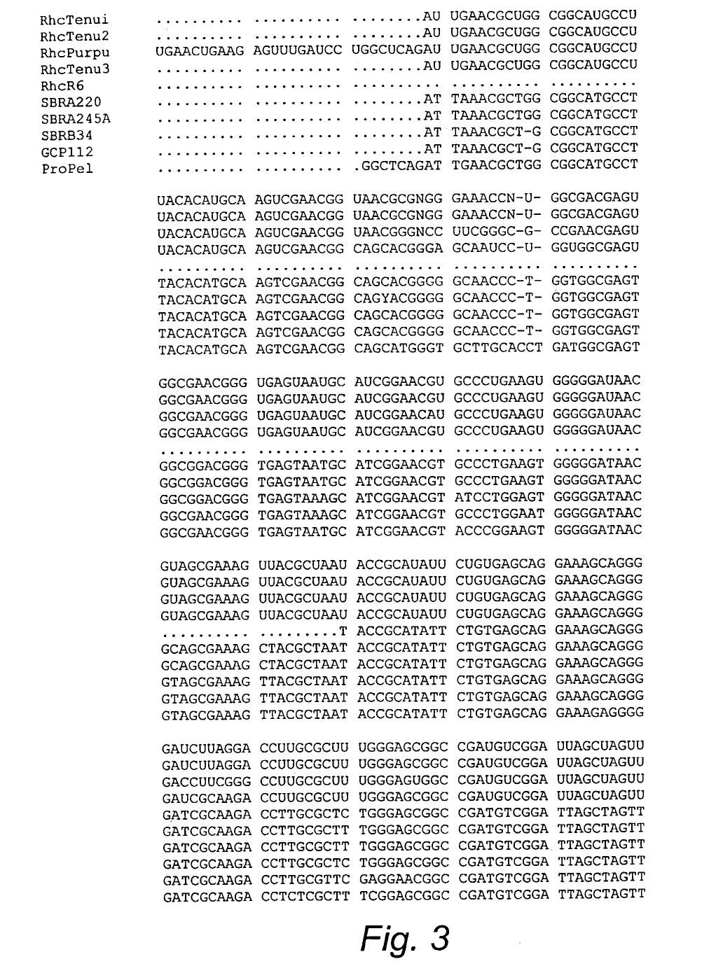

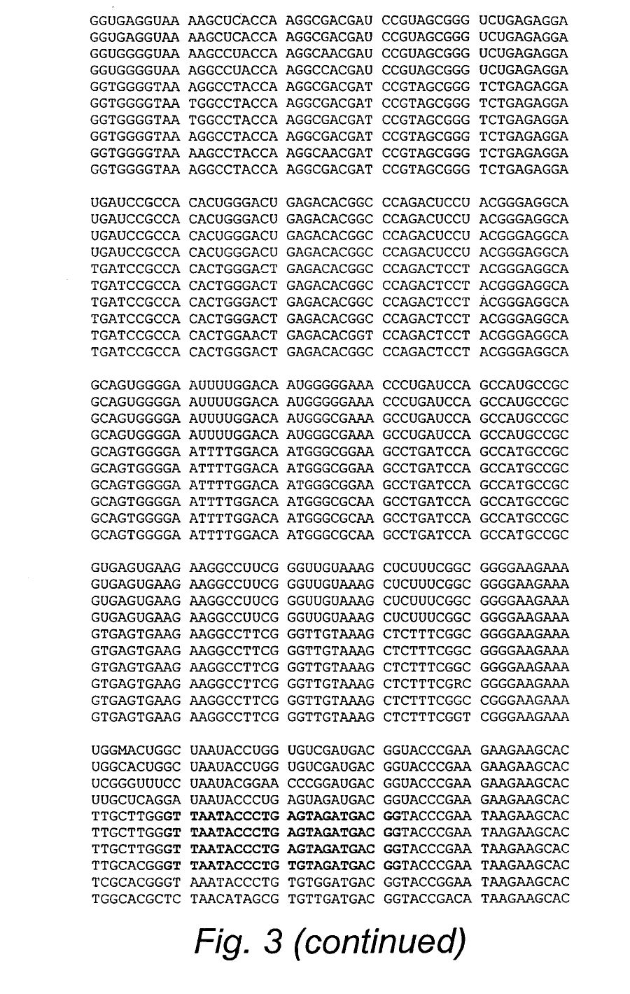

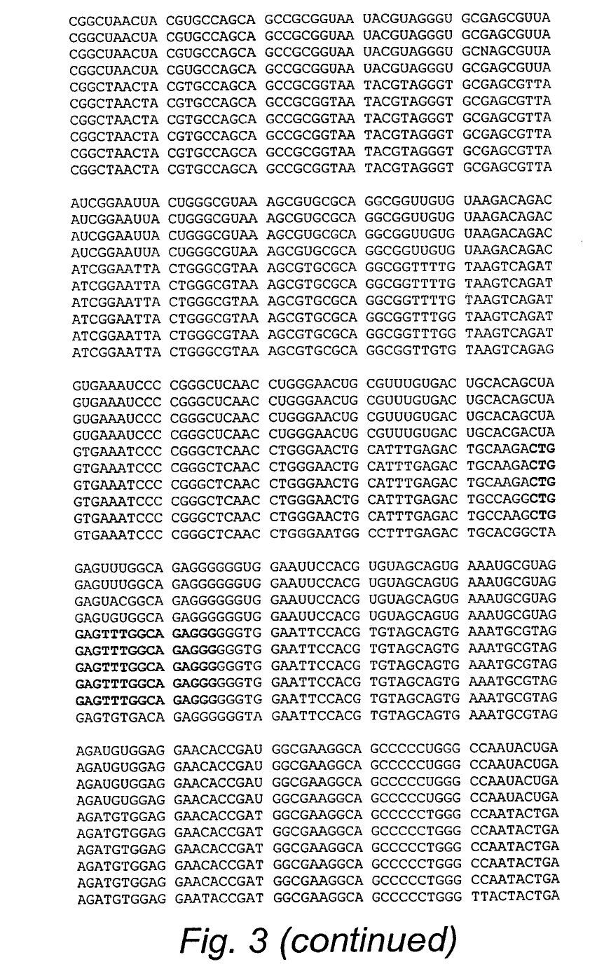

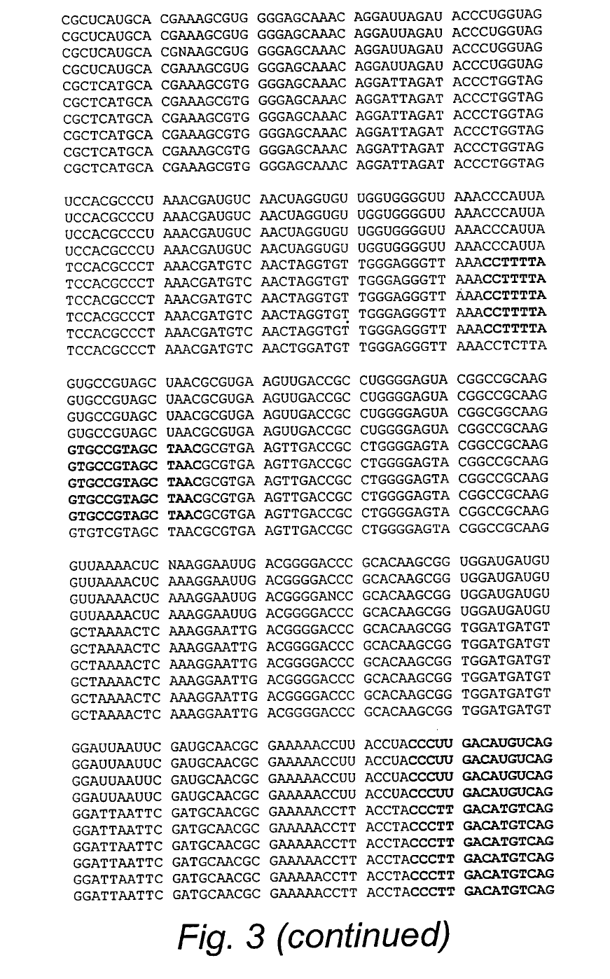

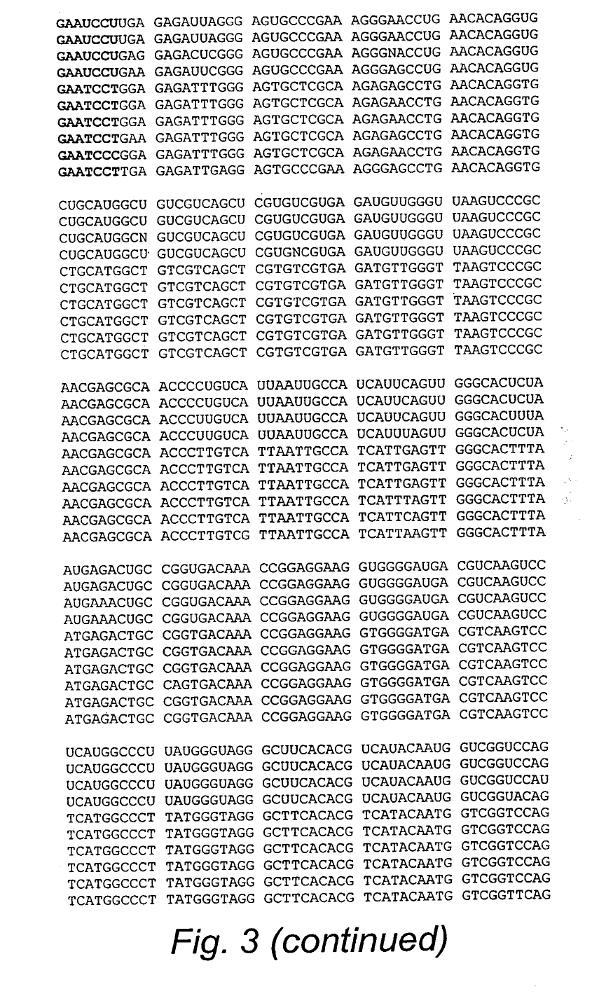

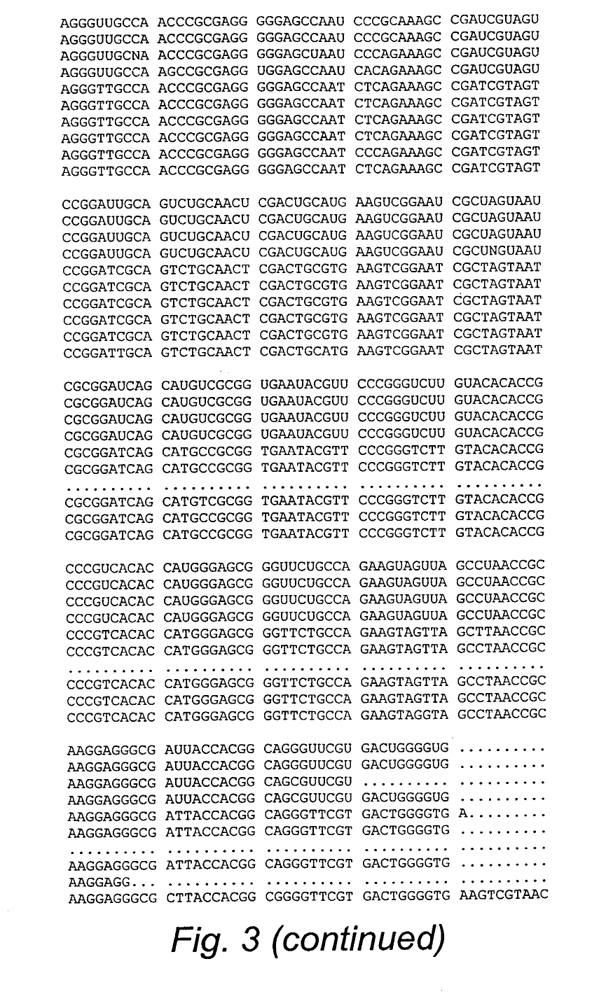

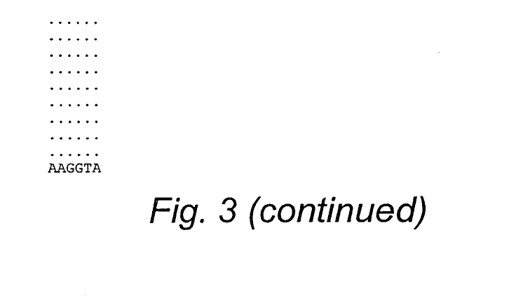

Other suitable sequences can be selected from the sequence alignment presented in FIG. 3.

There are a number of factors to be considered when designing primers and probes according to the invention. These factors will now be briefly discussed.

Specificity. Specificity is the first and foremost design Consideration for probes and primers. It is achieved by selecting a complementary sequence to the 16S rRNA and or 16S rDNA of a target organism with no mismatches (non-canonical base pairing). Non-target organisms must have at least one mismatch to the probe or primer sequence to ensure that hybridization will not occur. The optimal position of mismatches in a hybridisation probe is in the middle of the oligonucleotide and the optimal position of mismatches in a PCR primer is at the 3′ (extension) end. All probes of the present invention were designed for specificity using the ARB software package (Strnnk et al., unpublished). The following parameters were subsequently assessed after the initial design in ARB.

Thermodynamic considerations. The hybridization of probes or primers is dependent on physical parameters, the most important of which is temperature. Therefore, thermodynamic parameters of the probe or primer such as melting temperature (Tm) or dissociation temperature (Td) (Keller, 1993) determine the conditions under which the specific hybridization of the oligonucleotides will occur.

Accessability. In the case of FISH, according to the second embodiment of the invention, accessibility of the ribosome is an important design consideration(Fuchs et al., 1998). Some regions of the 16S rRNA within the ribosome are less accessible than others, in the worst case scenario preventing the access of oligonucleotides to those sites leads to no detection.

Secondary structure considerations Oligonucleotides can have self complementarity resulting in either dimer formation or hairpin structures. Secondary structures of the probe are an important design parameter when used with ion-channel membrane biosensors.

A primer according to the second embodiment of the invention, like the probes of the first embodiment, typically has a length of 12 to 50 nucleotides. A preferred length is 12 to 22 nucleotides.

Oligonucleotide primer pairs according to the third embodiment of the invention comprise an oligonucleotide primer that will anneal to one strand of the target sequence and a second oligonucleotide primer that will anneal to the other, complementary, strand of the target sequence. It will be appreciated that the second oligonucleotide primer must anneal to the complementary strand downstream of the first oligonucleotide primer sequence, which occurs in the complementary strand, to yield a double stranded amplification product in the PCR. The amplification product is of a size that facilitates detection. Typically, the first and second oligonucleotide primer sites in the target DNA are separated by 50 to 1,400 bps. A preferred separation is 400 to 1,000 bps.

As indicated above, probes according to the first embodiment of the invention can be used as hybridisation probes to detect PAOs. A preferred hybridisation technique is FISH for which the specific oligonucleotides specified above are particularly suited. However, the probes can be used in any other hybridisation technique as will be discussed below.

A method utilising probes according to the invention is defmed in the fourth embodiment. The probe label can be any label suitable for in situ detection of the probe-RNA hybrid. A preferred label is a fluorescent label such as fluorescein. A detailed description of the FISH technique is given in an article by De Long et al. (1989), full details of which are given in the reference listing.

In accordance with the fifth embodiment of the invention, probes of the first embodiment can be used in more general hybridisation techniques or in specialised techniques such as an ion-channel biosensor. Specifically, nucleic acid from a PAO can be immobilised on an inert support such as a membrane. After hybridisation of the probe to the immobilised nucleic acid, the hybrid is detected by virtue of the label. A particularly convenient hybridization technique makes use of a slot blot manifold such as the quantitative method described by Stahl et al. (1988).

Probes used in general hybridisation techniques can be longer than the typical length of up to 50 nucleotides In an ion channel biosensor, a probe is attached to an ion-channel membrane biosensor. When target 16S rDNA or rRNA binds to the probe, the ion-channel switch is triggered. The switching event results in a drop in electrical conductance and thereby indicates that target nucleic acid is present. The mechanism of the biosensor is described in detail in an article by Cornell et al. (1997).

The label of probes according to the invention can be any of the labels known to those of skill in the art. For example, the label can be a radiolabel, a reporter group or a hapten.

The method of the fifth embodiment can also be used to quantitate the number of PAO cells in a sample. With the more general hybridisation techniques, this is done by comparing the signal obtained from the probe-nucleic acid hybrid with a reference standard or a number of standards. That is, a standard is constructed comprising a known number of cells or a known amount of PAO DNA and the signal from the standard used to give a quantitative measure of the cells or DNA in the test sample. An ion-channel biosensor is particularly suited to quantitative determination of cell numbers as the drop in electrical conductance on triggering of the switching event gives a quantitative measure.

In the sixth embodiment of the invention, PCR is used to exponentially amplify 16SrDNA sequences using oligonucleotide primers. An example of its use in detecting microorganisms is given in Burrell et al. (1998). Detection of the amplified DNA can be by any of the methods known to those of skill in the art. For example, the amplified DNA can be analysed by agarose gel electrophoresis followed by staining to identify the DNA band of the expected size. Other methods for the detection of amplification products include hybridisation, especially solution hybridisation, using a labeled, internal oligonucleotide probe complementary to a region of DNA lying between the ends of the amplified DNA. The internal oligonucleotide can be labeled using any of the labels known to those of skill in the art. For example, the label can be a radiolabel or a non-radioactive label such as biotin. Nick-translation can also be used to label internal probes.

Probes and primers according to the invention can be prepared by conventional methods. Labeling can be done, if appropriate, during synthesis of the oligonucleotide constituting the probe or primer, or can be done post-synthesis. Methods for the labeling of primers is given in standard texts such as Sambrook et al. (1989).

Probes and primers can be provided as kits for use in the methods of the invention. A kit can include one probe or primer and appropriate reagents for carrying out the method. Advantageously, kits for PCR amplification of target DNA include at least one primer pair according to the third embodiment. In the case of a quantitative method, kits advantageously include at least one reference standard.

The methods of the invention allow quick and convenient assessment of whether a sludge or wastewater sample includes PAOs and also allow quantitation of the levels of PAO cells in samples. Thus, wastewater system managers can quickly diagnose any problems in the system due to PAO levels. Kits according to the invention are particularly useful in this regard.

To develop PAO specific probes and primers, sequence information is required. A panel of PAO 16S rDNA sequences and sequences of 16S rDNA from other organisms must be constructed. From the panel, sequences unique to PAO 16S rDNA can be selected. The sequence alignment of FIG. 3 constitutes a particularly suitable panel for the identification of sequences unique to PAOs

The general techniques used in the various embodiments of the invention will be known to those skilled in the art. Such techniques are described, for example, in Sambrook et al. (1989).

A non-limiting example of the invention follows.

### EXAMPLE 1

Development of Probes for Detecting Polyphosphate Accumulating Organisms

In this example we described how various sludges were enriched for polyphosphate accumulating organisms, the preparation and characterisation of 16S rDNA clone libraries from these sludges, and the development of FISH probes.

1.1 Methods

Generation of Sludges Enriched with Polyphosphate Accumulating Microorganisms

Two sludges were generated in Brisbane, Queensland, Australia (A and GRC sludges) and one was generated in San Francisco, Calif., USA (B sludge). The reactor and media used for the A and the GRC sludges and the methods for their evaluation are the same as those reported by Bond et al. (1999a). Briefly, a 1.8 to 2 liter sequencing batch reactor (SBR) was operated in anaerobic/aerobic cyclic conditions for enhanced biological phosphorus removal (EBPR) using a synthetic wastewater mix (Bond et al., 1999a). The SBR was fitted with pH electrodes and a portable dissolved oxygen electrode, and a 6h operating cycle consisting of 2-h anaerobic, 3.5-h aerobic and 0.5-h settling, was maintained. A hydraulic retention time (HRT) of 12 h was maintained as 900 mL or 1 liter of media was fed in the first 10 min of the anaerobic period, and 900mL or 1 liter of treated-supernatant was withdrawn in the last 5 min of the settling stage. Mixed liquor was wasted during the aeration period so that the solids retention time (SRT) was 8 to 10 d.

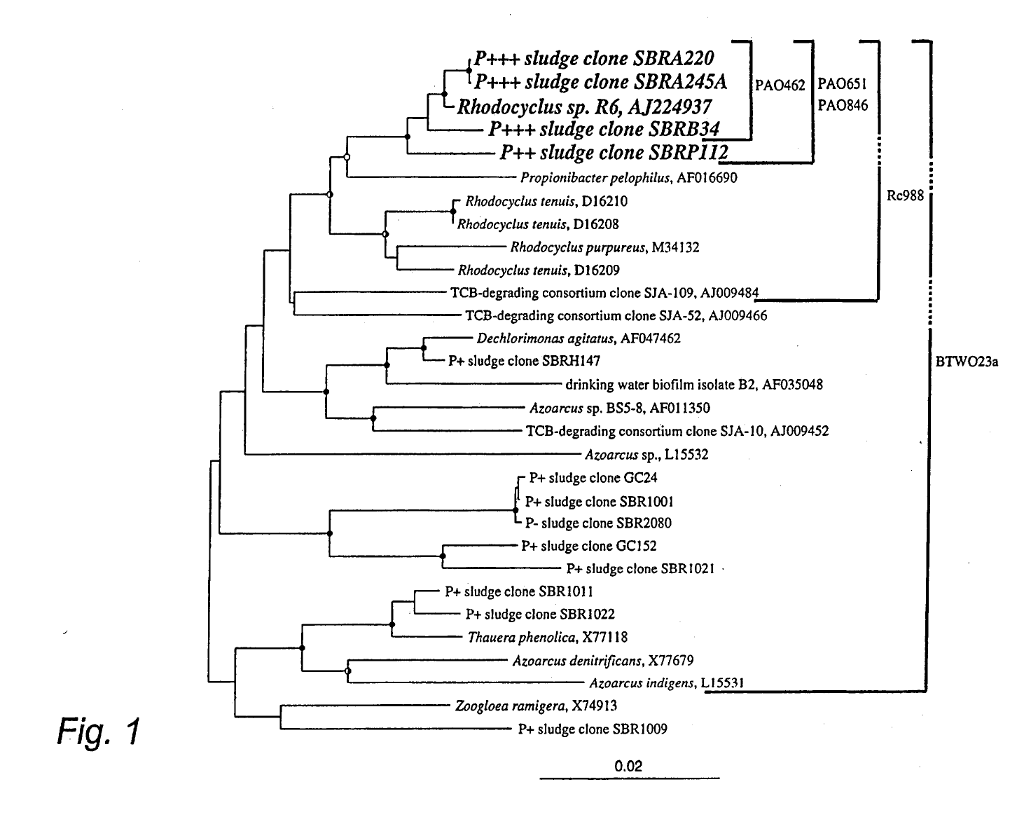

The PO₄-P concentration in the influent to the A sludge was increased to 57 mg PO₄-P/liter, while that in the GRC sludge was 28 mg/liter. The effluent PO₄-P concentration inthe A sludge was always at or below the detection limit (0.05 mg PO₄-P/liter). At this point, the P% of the mixed A culture was 15.1%. The performance of the GRC reactor fluctuated over a 12 month period and at regular stable operating times, the sludge was analysed by FISH and the P% determined. Images presented in FIG. 1 from the GRC sludge were when the reactor effluent was 6.7 mg PO₄-P/liter and the sludge contained 6.7% P.

Reactor B was also operated as an SBR with a working volume of 1 liter, a temperature of 23.5° C.±2°, and the pH was controlled in the range 7.15-7.25 by the addition of either a 1% HCl or a 40 g/liter Na₂CO₃ solution. The 6-h cycle consisted of 1.83-h anaerobic, 3-h aerobic, 0.5-h, settle, and 0.67-h comprising draw, fill, and strip with nitrogen gas. An HRT of 12 h was maintained by withdrawing 500 mL of the reactor contents during each settle phase and replacing it with 500 mL fresh nutrient feed. Timed operation of feed and effluent pumps, air and nitrogen flow, and mixing was by a programmable controller (Model CD-4, Chrontrol Corp., San Diego, Calif.). The SRT was maintained at 4 d (25% of the biomass wasted/d) by once per day manually withdrawing a portion of the mixed reactor contents immediately prior to the settle phase during the same cycle. The sludge in the reactor had a P% of 17.2%.

Anaerobic conditions were maintained by continuous bubbling with N₂ gas through a porous diffuser. N₂-stripping of oxygen began approximately 30 min before the addition of feed. Aerobic conditions were maintained by bubbling ambient air through a porous diffuser. Air and N₂ flow rates were approximately 500 mljmin. Anaerobic and aerobic conditions were verified by continuous measurements using an in-reactor oxygen electrode (M1016-0770, New Brnnswick Scientific, Edison, N.J.), a dissolved oxygen meter (Model DO-40, New Brunswick Scientific) and a strip chart recorder (Model 288, Rustrak Corporation, Manchester, N.H.).

Nutrient and carbon feeds were added separately. The nutrient feed consisted of (per liter) 259 mg NaH₂PO₄•2H₂O (50 mg P/liter), 117 mg KCl, 119 mg NH₄Cl, 219 mg MgCl₂•6H₂O, 14.4 mg MgSO₄•7H₂O, 45.9 mg CaCl₂, 8.3 mg yeast extract, 0.24 mL 10% HCl, 0.20 mL trace element solution, and 0.15 mL FeSO₄ solution. The trace element solution consisted of (per liter) 300 mg H₃BO₃, 1 500 mg ZnSO₄•7H₂O, 75 mg KI, 300 mg CuSO₄•5H₂O, 367 mg Co(NO₃)₂•6H₂O, 150 mg Na₂MoO₄•2H₂O, and 1,679 mg MnSO₄•H₂O. The FeSO4 solution was 2,054 mg/liter FeSO₄•7H₂O. The carbon feed was added as a concentrated stock (10 mL per cycle). The carbon feed consisted of 425 mg CH₃COONa•3H₂O and 30 mg casamino acids per liter of nutrient feed.

The reactor was seeded with mixed liquor from the City of San Francisco Southeast Water Pollution Control Plant (SEP) which is a pure-oxygen activated sludge plant with six basins in series, the first of which functions as an anaerobic selector. High soluble P concentrations in the anaerobic selector were an indication of the presence of EBPR organisms in the SEP.

Reactor analyses. Performance of all three reactors (A, GRC, and B) was superficially assessed by determination of the supematant P and acetate concentrations at the end of the anaerobic and aerobic periods, by the effluent P concentration, and by the sludge P%. P and acetate concentrations were also determined in each batch of feed prepared. At weekly or biweekly intervals during the operation of the reactors, cycle studies were conducted. Samrples were collected from the reactor at 0.5-h intervals during one discrete cycle for determining supernatant acetate and P concentrations, and cellular carbohydrate and polyhydroxyalkanoate (PHA) content. For the A and GRC reactors, methods for analysis were as reported by Bond et al. (1999a) but procedures employed in the B reactor are reported below.

Chemical Analyses

Phosphate. Soluble orthophosphate (Psol) was on GF/B-filtered (P/N 1821025, Whatman International, Ltd., Maidstone, UK) or 0.45 μm membrane filtered (P/N 60172, Gelman Sciences, East Hills, N.Y.) samples by the vanado-molybdate colorimetric method (Method 4500-P C; APHA et al., 1992). Total orthophosphate (Pt) was by the persulfate digestion method (Method 4500-P B.5; APHA et al., 1992). Non-soluble phosphate (Pns) was calculated as (Pt-Psol) for samples taken at the end of the aerobic phase.

Acetate. Acetate was analyzed on filtered (GF/B or 0.45 Fm membrane filters) acidified samples by flame ionization detector gas chromatography (FID GC), using a J&W Scientific DB-FFAP 0.53 mm capillary column. Samples were acidified with concentrated H₃PO₄ and stored at 4° C. prior to analysis when 2 μL samples were injected. The carrier gas was N₂ with a flow rate of 15 mL/min; H₂ flow rate was 20 mL/min and the air flow rate was 250 mL/min to the FID. Oven temperature began at 90° C. ramped to 110° C. at 50° C./min, remained at 110° C. for 30 s, and then ramped to 130° C. at 50° C./min. Injector temperature was 250° C.; the FID was unheated.

Polyhydroxyalkanoates. PHAs were determined by a modification of the GC method of Riis and Mai (1988) as follows: 10 mL samples were collected on 25 mmWhatman GF/B filters and immediately dried at 100° C. for 1 h then stored in a desiccator at 4° C. prior to analysis; 1 mL of 4:1 l-propanol:HCl and 1 mL of trichloroethene were added to each sample in 10 mL sample vials, which were then capped and heated to 95-100° C. for 3-4 h. Samples were cooled and then extracted with 2 mL deionized water. PHAs in the lower phase were measured by injection of 2 μL into an FID GC (glass packed column, 10% AT-1000 resin on Chromosorb W-AW 80-100 mesh, Varian model 3700 GC). Samples of 2 μL volume were analyzed using the following temperatures: oven, 250° C.; injection port, 250° C.; FID, 220° C. Benzoic acid was used as an internal standard.

Carbohydrates (CH). Total CH was by the anthrone method described in Jenkins et al. (1993) with the following modifications. Samples were diluted to 1 mL in 15 mL test tubes and frozen until analysis. Dilution water was pre-frozen in the test tubes to rapidly stop metabolic activity. Soluble CH was measured on Whatman GF/B-filtered samples. Duplicate glucose standard samples were analyzed with each batch of samples.

Total suspended solids (TSS) and volatile suspended solids (VSS). TSS and VSS were by Standard Methods 2540B and 2540E, respectively (APHA et al., 1992).

Microbiological Analyses

Microscopy of Mixed Cultures. Mixed cultures (sludges) from the A, GRC and B reactors and from other reactors were collected, fixed and probed as reported by Bond et al. (1999a). Counting of the probed A sludge was done manually and occasionally, this mixed microbial culture required light sonication (Bond et al., 1999) to facilitate the process. Counts of α, β (including β-1 and β-2), and γ-Proteobacteria, Actinobacteria, and Cytophaga-Flavobacterium were determined as proportions of all Bacteria (according to probe EUB338; Bond et al., 1999a—see below for details of probes) for the A sludge. Methylene Blue and Gram stains (Bond et al., 1999a) were also done on the A and GRC sludges and on other selected sludges. For the B sludge, Neisser staining was as described in Eikelboom and van Buijsen (1981), Gram staining was by the Modified Hucker Method and India Ink staining were from Jenkins et al. (1993), and PIHB staining was as described in Murray (1981). Light micrographs of Gram and Methylene Blue stains were captured on a NikonMicrophot FXA microscope via a charged couple device connected to a PC. Images were prepared in Adobe Photoshop. FISH probed samples were viewed on both a Zeiss LSM510 and on a Zeiss Axiophot. The Zeiss LSM 510 confocal laser scanning microscope employed an Axiovert lOOM SP inverted optical research microscope, and a Plan-Neofluar 63×/1.25 numerical aperture objective. Scan time was 31.8 s per frame and 4.48 Is pixel dwell time. An Argon laser 488 nm line and the HeNe 543 nm line was used for imaging. Frame size was 512×512 pixels. Images presented in FIG. 1 were taken with the LSM510 and prepared in Adobe Photoshop.

Clone libraries. Bacterial 16S rDNA clone libraries were prepared from genomic DNA extracted from frozen A, P (Bond et al., 1999) and B sludges and inserts from individual clones were amplified and grouped according to restriction fragment length polymorphism (RFLP) analysis using methods previously described (Burrell et al., 1998). Clones of RFLβ-group representatives were partially sequenced using primer 530f and phylogenetically analysed (Bond et al., 1995; Burrell et al., 1998). A selection of clone inserts was fully sequenced with a range of primers (Blackall, 1994). Phylogenetic analysis of the 16S rDNA sequences was performed as described previously (Dojka et al., 1998). Briefly, sequences were compiled using the software package SeqEd (Applied Biosystems, Australia). Each of the compiled sequences was compared to available databases using the basic local alignment search tool (BLAST; Altschul et al., 1990) to determine approximate phylogenetic affiliations. All clone sequences were examined with the CHECK_CHIMERA program (Maidak et al., 1999) to identify any chimeric sequences. The compiled sequences were aligned using the ARB software package (Strunk, et al., unpublished) and alignments were refined manually. Phylogenetic trees were constructed by carrying out evolutionary distance analyses on the 16S rDNA alignments, using the appropriate tool in the ARB database. The robustness of the tree topology was tested by bootstrap analysis, using neighbour-joining with the Kimura 2-parameter, and parsimony analysis (version 4.Ob2a of PAUP*; Swofford, 1999).

Probe Design, Synthesis and Use

PAO-specific probes were designed using the probe design tool in the ARB software package (Strunk et al., unpublished). Based on comparative analysis of all sequences in the database, the program selected specific regions within the putative-PAO sequences which allowed their discrimination from all other reference sequences. Sequences were subsequently confirmed for specificity using BLAST (Altschul et al., 1990). The designed oligonucleotides were synthesised and labelled at the 5′-end with the indocarbocyanine dye CY3 by Genset (France). These fluorescently-labelled probes were evaluated with paraformaldehyde fixed A sludge. The formamide concentration for optimum probe stringency was determined by performing a series of FISH experiments at 5% formamide increments starting at 0% formamide. Under all but the lowest stringency conditions, the morphologically distinct clusters of Methylene Blue positive coccobacilli were the only cells which bound the PAO-probes. Therefore, the optimum formamide concentrations were determined by reference to the coccobacillus clusters. This was necessary because there are no pure cultures whose 16S rRNA would bind the PAO-probes. A similar approach was employed by Erhart et al. (1997). Generally all three designed PAO-probes, PA0462, PA065 1 and PA0846 (see below), were applied to any one individual sample spotted on the slide.

Use of designed probes with other sludges. A range of sludges from laboratory scale processes and full-scale EBPR plants was collected, fixed and probed with the newly designed probes PA0462, PA065 1 and PA0846 after determining the formamide concentration for optimum probe stringency.

Slot Blot Hybridisation

Labeling of Probes. Oligonucleotide probes were labeled with digoxigenin-ddUTP, using the Digoxigenin (DIG) oligonucleotide 3′-end labeling kit, according to the manufacturer's instructions (Boehringer Mannheim, Mannheim, Germany). A standard 20 pl labeling reaction involved the addition of 100 pmole of unlabeled oligonucleotide to 4 μl 25mM CoCl₂, 50 U terminal transferase, 1 μl of 1 mM digoxigenin-11-ddUTP, and sterile distilled water (to a final volume of 20 μl). The labeling reaction was incubated for 15 min at 37° C. and then terminated by the addition of 1 μl of 20 mg/ml glycogen solution and 1 μl of 200 mM EDTA. The labeled oligonucleotide was precipitated by the addition of 0.1 volume 3 M sodium acetate and 3 volumes 100% ethanol followed by incubation at −70° C. for 30 min. Following centrifugation at 12,000 g for 5 min, the ethanol was removed and the pellet was washed with 50 μl of cold 70% ethanol. After brief centrifugation, the 70% ethanol was removed and the pellet was dried under vacuum. Finally, the labeled probe was resuspended in 20 μl of steril milliQ water. The yield of each labeling reaction was then estimated by spotting dilutions of the labelled control DNA (supplied by manufacturer) and the newly labelled probe onto a nylon membrane. Following chemiluminescent detection the yield of labelled probe could be estimated by comparison with the control.

Application of RNA to Membrane. All RNA samples were denatured by heating at 96° C. for 10 min. The denatured RNA samples were slotted in a 50 il volume onto a moistened positively charged nylon membrane (Boehringer Mannheim, Mannheim, Germany) using a PR648 slot blot apparatus (Hoefer Scientific Instruments, San Francisco, USA) under slight vacuum. The RNA samples were then immobilised on the nylon membrane by ultra violet irradiation for 5 min or baking at 80° C. for 1 hr. For quantitative hybridisations, a IO ng to 40 ng serial dilution of each denatured RNA target (including standard RNAs) was immobilised.

Hybridisations and Washes. The membranes were prehybridised for 2 hr at 40° C. in 5 ml of hybridisation buffer (DIG Easy Hyb; 5× SSC, 0.1% N-laurylsarcosine, 0.02% SDS and 1% blocking solution). Hybridisations were performed with 2-5 μl (0.1 μl of probe per slot; 10-fold excess) of probe in 5 ml of hybridisation buffer at 40° C. for 12-16 hr. All hybridisation steps were carried out in a Hybaid Mini 10 hybridisation oven (Hybaid, United Kingdom). The membranes were then washed twice for 15 min at the same temperature (40° C) in wash buffer containing 1× SSC (150 mM NaCl, 15 mM sodium citrate, adjusted to pH 7) and 1% SDS, followed by a 10 min wash at the determined T_{d} value for each probe.

Chemiluminescent Detection. Following hybridisation and stringency washing, membranes were rinsed for 5 min in a wash buffer containing 0.1 M maleic acid, 0.15 M NaCl and 0.3% Tween 20, adjusted to pH 7.5 with NaOH. To eliminate high background, membranes were blocked for 30 min in 25 ml of blocking solution which consisted of a maleic acid buffer (0.1 M maleic acid, 0. 15M NaCl, adjusted to pH 7.5) containing 1% Diploma skim milk powder. Following blocking, the membrane was incubated at room temperature for 30-60 min with 2 μl of anti-DIG-alkaline phosphatase solution (Boehringer Mannheim, Mannheim, Germany) in 20 ml of blocking solution. Membranes were washed twice for 15 min at room temperature in 25 ml of the wash buffer as described above and then equilibrated in 25 ml of detection buffer (0.1 M Tris—HCl, 0.1 M NaCl, pH 9.5) for 5 min. The chemiluminescent substrate, CSPD (Disodium 3-(4-methoxyspiro{1,2-dioxetane-3,2′-(5′-chloro)tricyclo[3.3.1.1^{3.7}]decan}-4-yl) phenylphosphate) (Boehringer Mannheim, Mannheim, Germay) was diluted 1/100 in detection buffer and each membrane was sealed in a hybridisation bag containing 1-2 ml of CSPD solution, and incubated at 37° C. for 5 min. The membrane was briefly removed from the hybridisation bag and blotted onto Whatman 3MM paper to remove excess CSPD solution and incubated for a further 15 min at 37° C. (after resealing into the hybridisation bag) to enhance the luminescent reaction. Membranes were then visualized using the LumiImager (Boehringer Mannheim, Mannheimn, Germany) and the level of chemiluminescent signal from each of the slots was quantified using -the LumiAnalyst software.

Quantitative Hybridisation Analysis. A slope value for each RNA serial dilution was generated by plotting chemiluminescent signal (BLU=Beohringer Light Units) versus ng RNA. Slope date was used to calculate the percentage of PAOs (%PAO) provided the slope had a regression coefficient greater than 0.85. The equation used to calculate %PAO in each sludge RNA sample was as follows:

θₓ=[(Pₓ/P_{c})×(P′ₓ/P′_{c})⁻¹]×100

where θₓ is the specific hybridisation percentage (the percentage of the RNA sample hydridising to probe x), P is the slope of the hybridisation of probe to sample RNA, P′ is the slope of the hybridisation of the probe to a pure, known control RNA (RNA transcript from a PAO clone), x is the specific probe, and c is the universal bacterial probe EUB338.

1.2 Results

A, GRC and B Sludge Operation

Reactor operating data are presented in Table 1. For the A and B sludges, some comparative data are also presented in Table 2. The comparative data are from a number of literature sources as indicated in the first column of the table.

TABLE 1
Operating data for the A, B, and GRC
laboratory-scale EBPR reactors

| | | Feed | | | End of Anaerobic | | Effluent | | |
|---|---|---|---|---|---|---|---|---|---|
| | PO₄—P | Acetate | | MLSS^{a} | PO₄—P | Acetate | PO₄—P | Acetate | P % in |
| Sludge | (mg/liter) | (mg/liter) | COD:P | (mg/liter) | (mg/liter) | (mg/liter) | (mg/liter) | (mg/liter) | Sludge^{b} |
| A | 57 | 309 | 9 | 3,692 | 144 | undetected | <0.05 | undetected | 15.1 |
| B | 53 | 425 | 4.3 | 1,160 | 110 | undetected | 28 | undetected | 17.2 |
| GCR | 28 | 389 | 18 | 3,070 | 77 | undetected | 6.7 | undetected | 6.7 |

^{a}MLSS—mixed liquor suspended solids
^{b}P % = (P_{T}-Pe/MLSS) × 100 (P_{T} = total sludge phosphate in mg/liter; Pe = phosphate in the effluent in mg/liter; MLSS inmg/liter)

TABLE 2
Stoichiometry of the transformations important
in Enhanced Biological Phosphorus Removal

| | | | Anaerobic Transformations (molar ratios) | | |
|---|---|---|---|---|---|
| | | | | Intracellular | |
| | Sludge P content | Phosphate | Acetate | carbohydrate | Intracellular PHA^{b} |
| Sludge origin | (% of dry mass) | released | uptake | consumed^{a} | units produced |
| B sludge (this study) | 17.2 | 8.1 | 6 | 0.8 | na^{c} |
| A sludge (this study) | 15.1 | 8.1 | 6 | 0.4 | 3.3 |
| Lab scale | 14.4-15.6 | 6.4-11.0 | 6 | na | na |
| continuous process | | | | | |
| (Wentzel et al. al., 1998) | | | | | |
| S sludge (Bond et al. 1999b) | 12.3 | 8.4 | 6 | 0.6 | 2.4 |
| Lab sludge (Liu et al., 1997) | 12.1 | 8.0 | 6 | 0.8 | 4.0 |
| Lab sludge (Liu et al., 1997) | 9.1 | 6.5 | 6 | 0.7 | 3.4 |
| EBPR model | — | 3 | 6 | 1 | 4 |
| (Arun et al., 1988)^{d} | | | | | |
| EBPR model | — | 6 | 6 | 1 | 4 |
| (Smolders et al., l994)^{d} | | | | | |
| P sludge (Bond et al., 1999a) | 8.8 | 5.6 | 6 | 1.2 | 3.9 |
| Sludge (Satoh et al., 1992) | 6.3 | 5.2 | 6 | 1.2 | 3.9 |
| Lab sludge (Liu et al., 1997) | 6.0 | 5.4 | 6 | 1.4 | 4.2 |
| T sludge (Bond et al., 1999b) | 2.0 | 0.3 | 6 | 2.6 | 4.4 |
| Q Sludge (Bond et al., 1999a) | 1.8 | 0.3 | 6 | 2.6 | 4.4 |
| Lab SBR | 1.8 | ˜0 | 6 | na | na |
| (Cech & Hartman, 1993) | | | | | |
| Lab sudge (Liu et al., 1997) | 1.5 | 0.3 | 6 | 2.5 | 5.5 |
| Non-EBPR Model | 13 | 0.0 | 6 | 2.5 | 5.5 |
| (Satoh et al., 1994)^{d} | | | | | |

^{a}Molar units are calculated as moles of glucose monomers.
^{b}PHA = poly-β-hydroxyalkanoates.
^{c}na = not available
^{d}Bolded data are from theoretical ratios for EBPR models from Arun et al. (1988) and Smolders et al. (1994), and from Satoh et al. (1994 for non-EBPR model.

The laboratory scale systems were good models of EBPR processes. Table 1 shows that all three SBRs were performing EBPR since there was P release and acetate uptake by the biomass during the initial anaerobic stage. This can be appreciated by comparing PO₄-P and acetate data in the feed and at the end of the anaerobic stage (Table 1). During the subsequent aerobic period, all sludges took up excessive amounts of P, as seen by comparing the PO₄-P values at the end of the anaerobic stage with those from the effluent. A and B sludges were hyper-P removing with the sludges containing >15% PO₄-P which equates to ca. 50% inorganic polyphosphate. The GRC sludge was a good P removing sludge being able to remove >20 mg/L of PO₄-P from the wastewater (compare 28 mg PO₄-P/L in the influent with 6.7 mg/L in the effluent) and contained 6.7%P (Table 1).

The results presented herein show that the A and B sludges were able to remove more P than most previously reported sludges and contained among the highest P% of any prior art sludges (see Table 2). Only the sludge of Wentzel et al. (1988) compares with these two sludges and where the stoichiometric comparisons are available for all these sludges, the data are remarkably similar (Table 2).

Clone Libraries

A total of 281 bacterial 16S rDNA clones from the A sludge, 89 from the P sludge and 250 from the B sludge were evaluated by RFLP. These sludges were chosen to generate 16S rDNA sequences because they were high performance EBPR systems (Table 1) and therefore a good source of PAO sequences from which specific FISH probes could be designed. Group representatives were partially sequenced and the overall results are presented in Table 3.

TABLE 3
Proportions of the different major bacterial divisions
in the A, P, and B clone libraries as determined
by RFLP and sequencing of RFLP-group representatives

| | Clone | Clone | Clone |
|---|---|---|---|
| Bacterial Division or Subdivision | Library A | Library P | Library B |
| α Proteobacteria | 38 (14%) | 5 (6%) | 32 (13%) |
| β Proteobacteria (mostly | 13 (5%) | 15 (17%) | 44 (18%) |
| Rhodocyclus relatives) | | | |
| Actinobacteria (mostly Terrabacter | 67 (24%) | 8 (9%) | 22 (9%) |
| relatives) | | | |
| Cytophaga-Flavobacterium group | 83 (30%) | 45 (51%) | 52 (21%) |
| Total clones in the library | 281 | 89 | 250 |
| analysed by RFLP | | | |

Probe Development

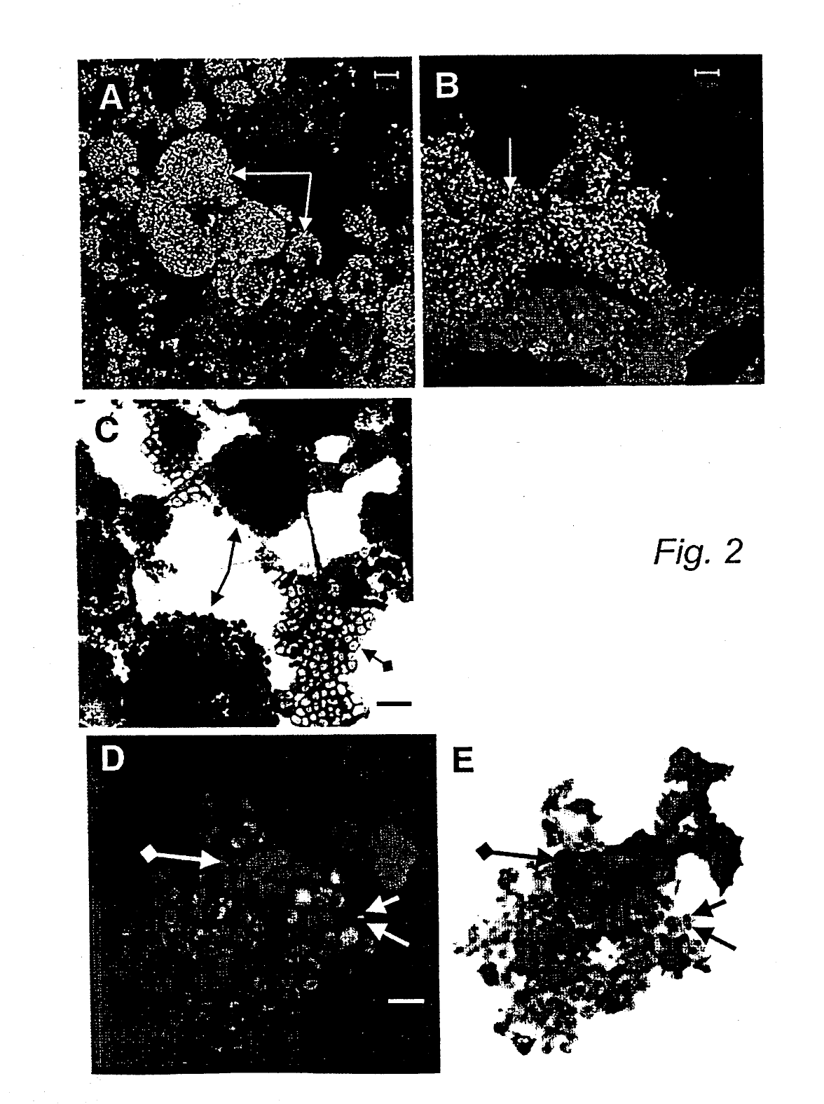

Group probing experiments were conducted using a number of known FISH probes. Details of these probes are included in Table 4. Table 5 includes the group probing results from the A sludge and a number of other sludges of various P-removal capacities. β Proteobacteria, specifically β-2 proteobacteria, dominated the A sludge community strongly suggesting the PAOs are members of this bacterial subdivision. In all cases, quantification of group probings of the GRC sludge was not performed but FIG. 2C (see below) shows the result from its methylene blue staining. EBPR sludges microscopically examined included those from the Loganholme Sewage Treatment Plant (full scale) and many laboratory scale reactors operated by researchers at the Advanced Wastewater Management Centre (A, P, GRC, Saline EBPR and denitrifing EBPR sludges; see Table 2 for the sources of these sludges). In all these EBPR sludges, the clusters of PAO-probe binding cells were distinct and uniform and resembled cells discussed below in connection with FIGS. 2A and 2C.

TABLE 4
Information relevant to FISH probes used in this study

| | | rRNA target | | Percent | |
|---|---|---|---|---|---|
| Probe | Sequence (5′-3′) | site^{a} | Specificity | formamide | Reference |
| EUB338 | GCTGCCTCCCGTAGGAGT | 16S, 338-355 | Bacteria | 20 | (Amann et |
| | | | | | al., 1990) |
| ALF1b | CGTTCG(C/T)TCTGAGCCAG | 16S, 19-35 | α Proteobacteria | 20 | (Manz et al., 1992) |
| BET42a | GCCTTCCCACTTCGTTT | 23S, 1027-1043 | β Proteobacteria | 35 | (Manz et al., 1992) |
| BONE23a | GAATTCCATCCCCCTCT | 16S, 663-679 | β-1 Proteobacteria | 35 | (Amann et |
| | | | | | al., 1996) |
| BTWO23a | GAATTCCACCCCCCTCT | 16S, 663-679 | competitor for BONE23a | 35 | (Amann et |
| | | | | | al., 1996) |
| GAM42a | GCCTTCCCACATCGTTT | 23S, 1027-1043 | γ Proteobacteria | 35 | (Manz et al., 1992) |
| HGC69a | TATAGTTACCACCGCCGT | 23S, 1901-1918 | Actinobacteria | 25 | (Roller et al., 1994) |
| CF319 | TGGTCCGTGTCTCAGTAC | 16S, 319-336 | Cytophaga-Flavobacterium | 35 | (Manz et al., 1992) |
| PAO462 | CCGTCATCTAC(A/T)CAGGGTATTAAC | 16S, 462-485 | PAO cluster (see FIG. 1) | 35 | This study |
| PAO651 | CCCTCTGCCAAACTCCAG | 16S, 651-668 | PAO cluster (see FIG. 1) | 35 | This study |
| PAO846 | GTTAGCTACGGCACTAAAAGG | 16S, 846-866 | PAO cluster (see FIG. 1) | 35 | This study |
| Rc988 | AGGATTCCTGACATGTCAAGGG | 16S, 988-1009 | “Rhodocyclus group” | nd^{b} | This study |
| | | | (see FIG. 1) | | |

^{a}rRNA Escherichia coli numbering (Brosius et al., 1981).
^{b}nd = not determined

TABLE 5
Bacterial community analysis of EBPR
sludges from laboratory scale SBRs

| Group according to FISH | Q sludge^{a} | T sludge^{b} | P sludge^{a,b} | S sludge^{b} | A sludge^{c} |
|---|---|---|---|---|---|
| probing | (1.8% P) | (2.0% P) | (8.8% P) | (12.3% P) | (15.1% P) |
| α Proteobacteria | <1 | 42 | 4-10 | 9 | 12 |
| β Proteobacteria | 58 | 13 | 42-45 | 56 | 80 |
| β-1 Proteobacteria | <1 | nd^{d} | 2 | nd | 1 |
| β-2 Proteobacteria | <1 | nd | 55 | nd | 81 |
| γ Proteobacteria | <1 | 16 | ca. 1 | 2 | 1 |
| Actinobacteria | <1 | 40 | 35-43 | 35 | 28 |
| Cytophaga- | Nd | 6 | 12 | 9 | 14 |
| Flavobacterium | | | | | |

^{a}Bond et al. (Bond et al., 1999a)
^{b}Bond et al. (Bond et al., 1999b)
^{c}this study
^{d}nd = not determined

As noted above, the group probing broadly highlighted the PAOs as β-2 Proteobacteria (see Table 5). However, the β-2 Proteobacteria probe (BIWO23a) was originally designed only as a competitor for the β-1 Proteobacteria probe (BONE23a; Amann et al., 1996). Its specificity is broad since it targets (with no mismatches) members of the β-3 Proteobacteria, some γ Proteobacteria and a Green-non-sulfur division clone, OPB9, in addition to P2 Proteobacteria. Therefore, additional more-specific probes were required to target the β-2 Proteobacteria group. To this end, all clones from the A, P and B sludge libraries belonging to the β-2 Proteobacteria were fully sequenced in preparation for probe design. In addition, partially sequenced clones belonging to the β-2 Proteobacteria from two previously reported EBPR and non-EBPR clone libraries (Bond et al., 1995) and sludge clone SBRH147 from an unpublished library were fully sequenced.

It is to be noted that the relative proportions of phylogenetic groups in the A sludge clone library (see Table 3) did not match those determined by FISH probing (see Table 5). The inventors recognise that clone libraries may not provide quantitative data on the microbial community structure of the sample analysed. Indeed, this highlights the need for specific probes for PAOs.

FIG. 1 shows a phylogenetic tree of the near complete sequenced β-2 Proteobacteria clones from which the PAO probes were designed, and the specificity of the probes. The 16S rDNA sequences were determined from sludges A, B, P, SBRH, SBR1, SBR2 and GC (Gold Coast, Queensland, Australia). The other sequences were obtained from publically accessible databases. Rubrivivax gelatinosus (D16213) was used as the outgroup in analyses but is not shown in the tree. Evolutionary distance and parsimonious analyses were carried out in PAUP* employing 2000 bootstrap resamplings. Closed circles at nodes indicate >75% bootstrap support from both analyses; open circles, 50-75% from both analyses; and half shaded circles are for analyses where one algorithm gave >75% bootstrap support and the other 50-75%. The coding P+++ indicates the clone came from a hyper-P removing sludge (ca. 15%P in the sludge); P++, a good P removing sludge; P+, a fair P removing sludge; and P−, a non-P removing sludge. The specificity of the published β-2 Proteobacteria probe (BTWO23a) and those of probes designed in this work (PAO-probes and Rc988) is shown by solid lines. Dashed lines against sequences indicate that the probe does not have 100% identity with that sequence. For example, Rc988 has one mismatch (at position 1009) with SBRP 112 sequence. In addition to specifically targeting the sequences indicated in the tree, the probe BTW023a also targets (with no mismatches) members of the β-3 Proteobacteria, γ Proteobacteria; Iodobacter spp., Chromobacterium spp., Chromatium spp. and a Green-non-sulfur division clone, OPB9. The scale indicates 0.02 nucleotide changes per nucleotide position.

Two main clusters of EBPR sludge clones were observed (SBRA220 cluster and GC4 cluster, FIG. 1). However, only the SBRA220 cluster was comprised exclusively of clones from high performance EBPR sludges. This became the focus group for probe design. Three PAO-probes were designed to specifically target the SBRA220 cluster and an additional probe of broader specificity called Rc988 (Table 5) was designed. All PAO-probes are listed in Table 4 with their empirically determined optimum stringencies.

Near complete 16S rDNA sequences for the hyper-P removing sludge clones P+++ SBRA220, P+++ SBRA245A, P+++ SBRB34 and P+++ SBRP112 and other sequences are presented as an alignment in FIG. 3. The reverse complement of the PAO probes and the Rc988 probe derived from these sequences are highlighted in the figure.

Use of Designed Probes

A series of fixed sludges including the A sludge, the GRC sludge at different operational periods, and the Loganholme sludge were evaluated with the designed PAO-probes of Table 4.

FIGS. 2A and 2B show confocal laser scanning micrographs of sludges dual probed with EUB338 (25 ng, fluorescein-labelled) and a mixture of all three PAO probes (Table 4, each 25 ng, CY3 labeled). Images were collected for fluorescein and CY3 channels, artificially coloured and superimposed. Arrowed cells are the PAOs since they are dual labelled with EUB338 (grey-coloured cells) and PAO probes (bright coloured cells that appear white in the image). FIG. 2A shows a mixed liquor from SBR A with operating data as given in Table 1. FIG. 2B shows lightly sonicated mixed liquor from an EBPR SBR (ca. 10% P in the sludge) operating at 3.5% NaCl in a study of seafood processing wastewater.

FIG. 2C is a bright field micrograph of GRC sludge as operated according to data in Table 1. In FIG. 2C, cells were methylene blue stained which stains for polyphosphate. The arrowed cells in FIG. 2C are those with polyphosphate and their cellular size, morphology and arrangement match the bright cells in FIG. 2A. The cells indicated in FIG. 2C with an arrow having a diamond shaped tail do not contain polyphosphate. The length of the bar in FIG. 2C is 6 μm.

The micrographs shown in FIGS. 2D and 2E are of a single cluster of cells from the SBR A sludge that was first probed with the labelled PAO probes (FIGS. 2D) and then stained with methylene blue (FIG. 2E). The arrowed cells in FIG. 2D are the “bright cells” and were found to correspond to the cells stained with methylene blue in FIG. 2E which are again arrowed. Although difficult to determine from the montone reproduction of the micrographs, cells that did not bind the PAO probes were considerably darker and did not stain with the methylene blue. These cells are again indicated with an arrow having a diamond shaped tail. The bar in FIG. 2D represents 4 μm.

The FIGS. 2D and 2E results clearly show that the PAO probes are specific for polyphosphate accumulating organisms.

FIG. 2 shows that in all of the sludges, characteristic clusters of coccobacilli bound the PAO-probes and depending upon EBPR performance, greater or fewer clusters were present. For example, in the Loganholme sludge, a full-scale activated sludge plant treating domestic wastewater with an influent containing ca. 10 mg PO₄—P/liter, moderate numbers of clusters were observed. Large numbers of the clusters were observed in the hyper-β-removing systems like the A sludge (FIG. 2A). Light sonication of a laboratory-scale saline EBPR sludge was required for cell counting and this explains why the PAO-probe binding cells in FIG. 2B are not arranged in typical clusters. Nevertheless, in the saline sludge as in all sludges, the three PAO-probes bound the same cells as bound the β-2 Proteobacteria probe.

Experiments were also conducted to assess whether there is a correlation between the proportion of PAO-probe binding cells in a sludge sample and the sludge P%. The experiments comprised a FISH analysis conducted essentially as described above and a slot blot analysis. All three PAO probes were used in the FISH analysis while PAO-651 was used for slot blot hybridisation (see Table 4). The EUB338 probe was used as a measure of the total number of bacterial cells in the particular sample.

For the slot blot analysis, RNA transcripts were generated from several 16S rDNA clones one of which was SBRA220 (see above), for use as standards. The 16S rDNA inserts in an M13 vector were PCR-amplified using vector primers flanking the insert or the universal bacterial primer, 1492R. Purified PCR products were used as templates for in vitro transciption using either T7 or SP6 RNA polymnerase as appropriate. Purified RNA transcripts were estimated to have a size of approximately 1,500 bp, equivalent to 16S rRNA extracted from E. coli. The concentration of RNA in transcript preparations was 320-660 ng/μl.

As test samples, total RNA was extracted from activated sludge samples by lysis of cells, homogenation in the presence of highly denaturing guanidinuim isothiocyanate-containing buffer, and application of ethanolic homogenate to an RNeasy mini spin column. The concentration of RNA extracted from each of the samples was determined using the GeneQuant RNA/DNA calculator and was found to range from 50 ng/μl to 400 ng/μl.

Slot blot hybridisation was conducted as described above in section 1.1. RNA extracts were obtained from experimental reactor sludges (see above) and full-scale activated sludge samples collected from seven wastewater treatment facilities within south-east Queensland, Australia.

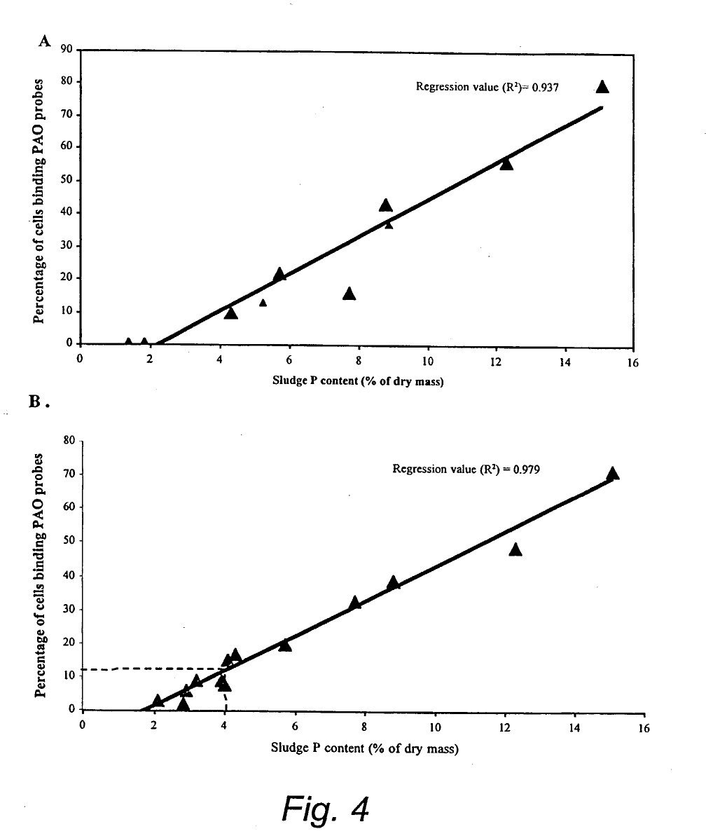

The FISH analysis was conducted on the GRC sludge at varying but stable β-removal efficiencies, the A sludge (see above), and the Q, P and S sludges of Bond et al. (Bond et al., 1999a; Bond et al., 1999b; Bond et al., 1998). The results of the FISH and slot blot analyses are presented in FIG. 4A and FIG. 4B, respectively. Large black triangles indicate results obtained using both methods while the grey triangles represent slot blot hybridisation results for the full-scale sludges. The small triangles in FIG. 4A are results for which there are no corresponding slot blot data.

FIG. 4 shows that there is a definite positive correlation between the proportion of PAO probe-binding cells and the sludge P%. The FISH analysis gave a regression value of 0.937 while an even high value of 0.979 was obtained with slot blot hybridisation. The FISH and slot blot evaluations were nevertheless comparable. The slot blot analysis also demonstrated that such a hybridisation technique can be used to accurately determine the proportion of PAOs in environmental RNA samples.

1.3 Utility of the PAO-Specific Probes

The PAO-probes, designed from a group of highly related clone sequences (greater than or equal to 98% identical) affiliated with the β-2 Proteobacteria, bound the same cell clusters in the A sludge, as bound the probe for β-2 Proteobacteria. The closest pure-cultured bacterial relatives to the β-2 Proteobacteria clone sequences (FIG. 1) are from Rhodocyclus (R. tenuis and R. purpureus) and Propionibacter pelophilus. A clone sequence from an as-yet-unpublished Swiss EBPR sludge (R6; Hesselmann et al., 1998) was in the group containing the full clone inserts from the A, P and B sludges (FIG. 1). Nearly 80% of the microbial biomass in the hyper-β-removing A sludge, bound the β Proteobacteria probe (BET42a) and all of these were β-Proteobacteria (Table 5) and PAO-probe positive. Thus, by using this concerted probing approach (see FIG. 1), it was demonstrated that the designed probes were highly specific for the dominant β-2 Proteobacteria in the A sludge. In addition, the PAO-probe positive cells matched the morphology, size and arrangement of those staining positive for polyphosphate by the methylene blue stain (FIG. 2). When used with other sludges, the PAO-probes and the β-2 Proteobacteria probe always bound the same cells. One demonstration of the simultaneous use of the three PAO-probes with another sludge is given in FIG. 2.

An indicative correlation between increasing P removal performance, as judged by P% in the sludge, and levels of p Proteobacteria was observed when data for the P sludge (8.8%P; 45% β Proteobacteria), the S sludge (12.3%P, 56% p Proteobacteria), and the A sludge (15.1%P, 80% β Proteobacteria) were compared (see Table 5). Data for Q, P, and A sludges specifically narrowed the β Proteobacteria to the β Proteobacteria (Table 5). When this correlation was more deeply investigated with the specific PAO-probes on the GRC, Q, P, S and A sludges, the link between P% in the sludge and numbers of PAO-probe binding cells was unequivocally demonstrated (FIG. 4). Clearly, the designed PAO-probes for particular β-2 Proteobacteria can be used to detect true PAO in sludge samples.

It will be appreciated by one of skill in the art that many different probes beyond those exemplified above can be prepared without departing from the broad ambit and scope of the invention.

References

Altschul, S. F., W. Gish, W. Miller, E. W. Myers, and D. J. Lipman. 1990. Basic local alignment search tool. J. Molec. Biol. 215:403-410.

Amann, R., J. Snaidr, M. Wagner, W. Ludwig, and K. H. Schleifer. 1996. In situ visualization of high genetic diversity in a natural microbial community. J. Bacteriol. 178:3496-3500.

Amann, R. I., B. J. Binder, R. J. Olson, S. W. Chisholm, R. Devereux, and D. A. Stahl. 1990. Combination of 16S rRNA-targeted oligonucleotide probes with flow cytometry for analyzing mixed microbial populations. Appl. Environ. Microbiol. 56:1919-1925.

Amann, R. I., W. Ludwig, and K. -H. Schleifer. 1995. Phylogenetic identification and in situ detection of individual microbial cells without cultivation. Microbiol. Rev. 59:143-169.

APHA, AWWA, and WPCF. 1992. Standard Methods for the Examination of Water and Wastewater, 18 th ed. Port City Press, Baltimore.

Arun, V., T. Mino, and T. Matsuo. 1988. Biological mechanism of acetate uptake mediated by carbohydrate consumption in excess phosphorus removal systems. Wat. Res. 22:565-570.

Blackall, L. L. 1994. Molecular identification of activated sludge foaming bacteria. Wat. Sci. Tech. 29(7):3542.

Blackall, L. L., S. Rossetti, C. Christenssen, M. Cunningham, P. HIartman, P. llugenholtz, and V. Tandoi. 1997. The characterization and description of representatives of “G” bacteria from activated sludge plants. Lett. Appl. Microbiol. 25:63-69.

Bond, P. L. 1997. PhD. The University of Queensland, Brisbane, Queensland.

Bond, P. L., R. Erhart, M. Wagner, J. Keller, and L. L. Blackall. 1999a. The identification of some of the major groups of bacteria in efficient and non-efficient biological phosphorus removal activated sludge systems. Appl. Environ. Microbiol. 65.

Bond, P. L., P. Hugenholtz, J. Keller, and L. L. Blackall. 1995. Bacterial community structures of phosphate-removing and non-phosphate-removing activated sludges from sequencing batch reactors. Appl. Environ. Microbiol. 61:1910-1916.

Bond, P. L., J. Keller, and L. L. Blackall. 1999b. Bio-P and non-bio-P bacteria identification by a novel microbial approach. Wat. Sci. Tech. 39 (6): 13-20.

Bond, P. L., J. Keller, and L. L. Blackall. 1998. Characterisation of enhanced biological phosphorus removal activated sludges with dissimilar phosphorus removal performance. Wat. Sci. Tech. 37:567-571.

Brosius, J., T. L. Dull, D. D. Steeter, and H. F. Noller. 1981. Gene organization and primary structure of a ribosomal RNA operon from Escherichia coli. J. Molec. Biol. 148:107-127.

Burrell, P. C., J. Keller, and L. L. Blackall. 1998. Microbiology of a nitrite-oxidizing bioreactor. Appl. Environ. Microbiol. 64:1878-1883.

Cech, J. S., and P. Hartman. 1993. Competition between polyphosphate and polysaccharide accumulating bacteria in enhanced biological phosphate removal systems. Wat. Res. 27:1219-1225.

Cloete, T. E., and P. L. Steyn. 1987. A combined fluorescent antibody-membrane filter technique for enumerating Acinetobacter in activated sludge, p. 335-338. In R. Ramadori (ed.), Biological Phosphate Removal from Wastewaters. Pergamon Press, Oxford.

Comeau, Y., K. J. Hall, R E. W. Hancock, and W. K. Oldham. 1986. Biochemical models for enhanced biological phosphorus removal. Wat. Res. 20:1511-1521.

Cornell, B. A., V. L. B. BraachMaksvytis, L. G. King, P. D. J. Osman, B. Raguse, L. Wieczorek, and R. J. Pace. 1997. A biosensor that uses ion-channel swiches. Nature. 387:580-583.

Deinema, M. H., M. C. M. van Loosdrecht, and A. Scholten. 1985. Some physiological characteristics of Acinetobacter spp. accumulating large amounts of phosphate. Wat. Sci. Tech. 17 (12):119-125.

DeLong, E. F., G. S. Wickham, and N. R. Pace. 1989. Phylogenetic stains: Ribosomal RNA-based probes for the identification of single cells. Science. 243:1360-1363.

Dojka, M. A., P. Hugenholtz, S. K- Haack, and N. R. Pace. 1998. Microbial diversity in a hydrocarbon and chlorinated-solvent-contaminated aquifer undergoing intrinsic bioremediation. Appl. Environ. Microbiol. 64:3869-3877.

Eikelboom, D. H., and H. J. J. van Buijsen. 1981. Microscopic Sludge Investigation Manual, 1^{st} ed. TNO Research Institute for Environmental Hygiene, The Netherlands.

Erhart, R., D. Bradford, R. J. Seviour, R. I. Amann, and L. L. Blackall. 1997. Development and use of fluorescent in situ hybridization probes for the detection and identification of “Microthrix parvicella” in activated sludge. Syst. Appl. Microbiol. 20:310-318.

Fuchs, B. M., G. Wallner, W. Beisker, I. Schwippl, W. Ludwig, and R. Amann. 1998. Flow cytometric analysis of the in situ accessibility of Escherichia coli 16S rRNA for fluorescently labeled oligonucleotide probes. Appl. Environ. Microbiol. 64:4973-4982.

Fuhs, G. W., and M. Chen. 1975. Microbiological basis of phosphate removal in the activated sludge process for the treatment of wastewater. Microb. Ecol. 2:119-138.

Hartley, K. J., and L. Sickerdick. 1994. Presented at the Second Australian Conference on Biological Nutrient Removal from Wastewater, Albury, Victoria.

Hesselmann, R., D. Hahn, J. R. van der Meer, and A. J. B. Zehnder. 1998. Erhohte biologische phosphatelimination aus abwasser. EAWAG News. 45:18-20.

Jenkins, D., M. G. Richard, and G. T. Daigger. 1993. Manual on the Causes and Control of Activated Sludge Bulking and Foaming. Lewis Publishers, New York.

Kampfer, P., R. Erhart, C. Beimfohr, J. Bohringer, M. Wagner, and R. Amann. 1996. Characterization of bacterial communities from activated sludge—culture-dependent numerical identification versus in situ identification using group- and genus-specific rRNA-targeted oligonucleotide probes. Microb. Ecol. 32:101-121.

Kawaharasaki, M., H. Tanaka, T. Kanagawa, and K. Nakamura. 1999. In situ identification of polyphosphate-accumulating bacteria in activated sludge by dual staining with rRNA-targeted oligonucleotide probes and 4′,6-diamidino-2-phenylindol (DAPI) at a polyphosphate-probing concentration. Wat. Res. 33:257-265.

Keller, G. H. 1993. Molecular hybridization technology, p. 1-25. In G. H. Keller and M. M. Manac (ed.), DNA Probes, 2nd ed. Stockton Press, New York.

Kerdachi, D. A., and K J. Healey. 1987. The reliability of cold perchloric acid extraction to assess metal-bound phosphates, p. 339-342. In R. Ramadori (ed.), Biological Phosphate Removal from Wastewaters. Pergamon Press, Oxford.

Liu, W.-T., K. Nakamura, T. Matsuo, and T. Mino. 1997. Internal energy-based competition between polyphosphate- and glycogen-accumulating bacteria in biological phosphorus removal reactors-effect of P/C feeding ratio. Wat. Res. 31:1430-1438.

Maidak, B. L., J. R. Cole, C. T. Parker, G. M. Garrity, N. Larsen, B. Li, T. G. Lilburn, M. J. McCaughey, G. J. Olsen, R. Overbeek, S. Pramanik, T. M. Schmidt, J. M. Tiedje, and C. R Woese. 1999. A new version of the RDP (Ribosomal Database Project). Nuc. Acids Res. 27:171-173.

Manz, W., R. Amann, W. Ludwig, M. Wagner, and IC-H. Schleifer. 1992. Phylogenetic oligonucleotide probes for the major subclasses of Proteobacteria: problems and solutions. Syst. Appl. Microbiol. 15:593-600.

Melasniemi, H., A. IIernesmaa, A. S.-L. Pauli, P. Rantanen, and M. Salkinojα-Salonen. 1999. Comparative analysis of biological phosphate removal (BPR) and non-BPR activated sludge bacterial communities with particular reference to Acinetobacter. J. Ind. Microbiol. 21:300-306.

Metcalf-and Eddy, I. 1991. Wastewater Engineering: Treatment, Disposal and Reuse. McGraw-Hill, New York.

Mino, T., M. C. M. van Loosdrecht, and J. J. Heijuen. 1998. Microbiology and biochemistry of the enhanced biological phosphate removal process. Wat. Res. 32:3193-3207.

Murray, R. G. E. 1981. Manual of Methods for General Bacteriology. American Society for Microbiology, Washington, D.C.

Nakamura, KV, A. Hiraishi, Y. Yoshimi, M. Kawaharasaki, K. Masuda, and Y. Kamagata. 1995. Microhenatus phosphovorus gen. nov., sp. nov., a new gram-positive polyphosphate-accumulating bacterium isolated from activated sludge. Int. J. Syst. Bacteriol. 45:17-22.

Riis, V., and W. Mai. 1988. Gas chromatographic determination of poly-b-hydroxybutyric acid in microbial biomass after hydrochloric acid propanolysis. J. Chromatog. 445:285-298.

Roller, C., M. Wagner, R. Amann, W. Ludwig, and I -H. Schleifer. 1994. In situ probing of Gram-positive bacteria with high DNA G+C content using 23S rRNA-targeted oligonucleotides. Microbiol. 140:2849-2858.

Sambrook, J., E. F. Fritsch, and T. Maniatis. Molecular Cloning: A Laboratory Manual, Second Edition, Cold Spring Harbor Laboratory Press, Plainview, N.Y., 1989.

Satoh, H., T. Mino, and T. Matsuo. 1994. Deterioration of enhanced biological phosphorus removal by the domination of microorganisms without polyphosphate accumulation. Wat. Sci. Tech. 30:203-211.

Satoh, H., T. Mino, and T. Matsuo. 1992. Uptake of organic substrates and accumulation of polyhydroxyalkanoates linked with glycolysis of intracellular carbohydrates under anaerobic conditions in the biological excess phosphate removal processes. Wat. Sci. Tech. 26:933-942.

Seviour, R. J., and L. L. Blackall. 1999. The Microbiology of Activated Sludge. Chapman and Hall, London.

Smolders, G. J. F., J. van der Meij, M. C. M. van Loosdrecht, and J. J. Heijnen. 1994. Model of the anaerobic metabolism of the biological phosphorus removal process—stoichiometry and pH influence. Biotechnol. Bioeng. 43:461-470.

Stahl, D. A., B. Flesher, H. R. Mansfield, and L. Montgomery. 1988. Use of phylogenetically based hybridization probes for studies of ruminal microbial ecology. Applied and Environmental Microbiology. 54:1079-1084.

Stante, L., C. M. Cellamare, F. Malaspina, G. Bortone, and A. Titche. 1997. Biological phosphorus removal by pure culture of Lampropedia spp. Wat. Res. 31:1317-1324.

Streichan, M., J. R. Golecki, and G. Schon. 1990. Polyphosphate-accumulating bacteria from sewage treatment plants with different processes for biological phosphorus removal. FEMS Microbiol. Ecol. 73:113-124.

Strunk, O., O. Gross, M. Reichel, S. May, S. Herrmann, N. Stuckmann, B. Nonhoff, M. Lenke, A. Ginhart, A. Vilgib, T. Ludwig, A. Bode, i-H. Scheiffer, and W. Ludwig. unpublished. ARB: a software environment for sequence data. URL: http://www.mikro.biologie.tumuenchen.de/.

Swofford, D. L. 1999. PAUP*: Phylogenetic Analysis Using Parsimony (version 4.Ob2a).

Tandoi, V., M. Majone, J. May, and R. Ramadori. 1998. The behaviour of polyphosphate accumulating Acinetobacter isolates in an anaerobic-aerobic chemostat. Wat. Res. 32:2903-2912.

Ubukata, Y. 1994. Some physiological characteristics of a phosphate removing bacterium isolated from anaerobic/aerobic activated sludge. Wat. Sci. Tech. 30-6:229-235.

van Loosdrecht, M. C. M., G. J. Smolders, T. Kuba, and J. J. Heijnen. 1997. Metabolism of microorganisms responsible for enhanced biological phosphorus removal from wastewater. Antonie van Leeuwenhoek. 71:109-116.

Wagner, M., R. Erhart, W. Manz, R. Amann, H. Lemmer, D. Wedi, and K -H. Schleifer. 1994. Development of an rRNA-targeted oligonucleotide probe specific for the genus Acinetobacter and its application for in situ monitoring in activated sludge. Appl. Environ. Microbiol. 60:792-800.

Wentzel, M. C., R. E. Loewenthal, G. A. Ekama, and G. v. Marais. 1988. Enhanced polyphosphate organism cultures in activated sludge systems—Part 1: Enhanced culture development. Water SA. 14:81-92.

Wentzel, M. C., L. H. Lotter, G. A. Ekama, R. E. Loewenthal, and G. v. R. Marais. 1991. Evaluation of biochemical models for biological excess phosphate removal. Wat. Sci. Tech. 23:567-576.

1. An oligonucleotide probe or primer for detecting a polyphosphate accumulating organism in a sample, said oligonucleotide having a sequence of 12 to 50 nucleotide selected from any one of SEQ ID NO. 5 to SEQ ID NO. 9 or the reverse complement of any one of SEQ ID NO. 5 to SEQ ID NO. 9; and wherein said oligonucleotide has the binding characteristics of an oligonucleotide of any one of the following sequences:

| | |
|---|---|
| 5′-CCGTCATCTACWCAGGGTATTAAC-3′ | (SEQ ID NO. 11) |
| 5′-CCCTCTGCCAAACTCCAG-3′ | (SEQ ID NO. 12) |
| 5′-GTTAGGTACGGCACTAAAAGG-3′. | (SEQ ID NO. 13) |

2. The oligonucleotide according to claim 1, wherein said oligonucleotide has a length of 15 to 25 nucleotides.

3. The oligonucleotide according to claim 1, wherein said oligonucleotide has a sequence selected from:

| | |
|---|---|
| 5′-CCGTCATCTACWCAGGGTATTAAC-3′ | (SEQ ID NO. 11) |
| 5′-CCGTGTGCCAAACTCCAG-3′ | (SEQ ID NO. 12) |
| 5′-GTTAGCTACQGCACTAAAAGG-3′. | (SEQ ID NO. 13) |

4. An oligonucleotide probe or primer for detecting organism in a sample related to polyphosphate accumulating organisms, said oligonucleotide having a sequence of 12 to 50 nucleotides selected from any one of the sequences of FIG. 3 (SEQ ID NO. 1 to SEQ ID NO. 10) or the reverse complement of any one of the sequences of FIG. 3; aid wherein said oligonucleotide has the binding characteristics of an oligonucleotide of the following sequence:
5′-AGGATTCCTGACATGTCAAAGGG-3′ (SEQ ID NO. 14).

5. The oligonucleotide according to claim 4, wherein said oligonucleotide have the following sequence:
5′-AGGATTCCTGACATGTCAAGGG-3′ (SEQ ID NO. 14).

6. A method of detecting cells of a polyphosphate accumulating organism in a sample, said method comprising the steps of:
(a) treating cells ill said sample to fix cellular contents;
(b) contacting said fixed cells from step (a) with a labelled oligonucleotide probe under conditions which allow said probe to hybridize with 16S rRNA within said fixed cell, wherein said probe is an oligonucleotide according to claim 1;
(c) removing unhybridized probe from said fixed cells; and
(d) detracting said labelled probe-RNA hybrid.

7. The method according to claim 6, wherein said label is a radiolabel, a reporter group or a hapten.

8. The method according to claim 6, wherein said detection is by fluorescence in situ hybridization.

9. A method of detecting a polyphosphate accumulating organism in a sample, said method comprising the steps of:
(a) obtaining nucleic acid from cells of said organism;
(b) contacting nucleic acid from step (a) wit a labelled or immobilised oligonucleotide probe under conditions which allow said probe to hybridize to 16S nucleic acid molecules, wherein said probe is an oligonucleotide according to claim 1;
(c) if necessary, separating unhybridized probe and labelled probe-nucleic acid hybrid; and
(d) detecting said labelled probe-nucleic acid hybrid.

10. The method according to claim 9, wherein said immobilization is to an inert support.

11. The method according to claim 9, wherein said detection is by an ion channel biosensor.

12. The method according to claim 9, comprising the further step of quantitating the number of cells of polyphosphate accumulating organism in said sample.
